(19) United States
(12) Patent Application Publication
(10) Pub. No.: US 2019/0284211 A1
(43) Pub. Date: Sep. 19, 2019

(54) CRYPTATES AND METHODS OF USE

(71) Applicant: NESTEC S.A., Vevey (CH)

(72) Inventors: Michael Hale, San Diego, CA (US); Darryl Rideout, San Diego, CA (US); Raj Srikrishnan, San Diego, CA (US); Stefan Westin, San Diego, CA (US); David Schmit, Lino Lakes, MN (US)

(73) Assignee: NESTEC S.A., Vevey (CH)

(21) Appl. No.: 16/404,455

(22) Filed: May 6, 2019

Related U.S. Application Data
(63) Continuation of application No. PCT/IB2018/050212, filed on Jan. 12, 2018.
(60) Provisional application No. 62/445,566, filed on Jan. 12, 2017.

(51) Int. Cl. C07F 5/00; C07D 487/08; G01N 33/542; G01N 21/64

(52) U.S. Cl. CPC C07F 5/00; C07D 487/08; G01N 2021/6439; G01N 21/6428; G01N 33/542

(57) ABSTRACT

The present invention provides cryptates with excellent fluorescent properties. The cryptates are useful in biological assays and methods for the detection and identification of various analytes.

### CROSS-REFERENCE TO RELATED APPLICATIONS

The present application is a continuation of PCT/IB2018/050212, filed Jan. 12, 2018, which application claims priority to U.S. Patent Application No. 62/445,566, filed Jan. 12, 2017, the teachings both of which are hereby incorporated by reference in their entireties for all purposes.

### BACKGROUND OF THE INVENTION

Cryptates are complexes that include a macrocycle within which a lanthanide ion such as terbium or europium is tightly embedded or chelated. This cage like structure is useful for collecting irradiated energy and transferring the collected energy to the lanthanide ion. The lanthanide ion can release the energy with a characteristic fluorescence.

Cryptates can be used in various bioassays formats. Some assays rely on time-resolved fluorescence resonance energy transfer (TR-FRET) mechanisms where two fluorophores are used. In these assays, energy is transferred between a donor fluorophore and an acceptor fluorophore if the two fluorophore are in close proximity to the each other. Excitation of the donor (cryptate) by an energy source (e.g. UV light) produces an energy transfer to the acceptor if the two fluorophores are within a given proximity. In turn, the acceptor emits light at its characteristic wavelength. In order for TR-FRET to occur, the fluorescence emission spectrum of the donor molecule must overlap with the absorption or excitation spectrum of the acceptor chromophore. Moreover, the fluorescence lifetime of the donor molecule must be of sufficient duration to allow the TR-FRET to occur.

U.S. Pat. No. 6,406,297 is titled “Salicylamide-lanthanide complexes for use as luminescent markers.” This patent is directed to luminescent lanthanide metal chelates comprising a metal ion of the lanthanide series and a complexing agent comprising a salicylamidyl moiety.

U.S. Pat. No. 6,515,113 is titled “Phthalamide lanthanide complexes for use as luminescent markers.” This patent is directed to luminescent lanthanide metal chelates comprising a metal ion of the lanthanide series and a complexing agent comprising a phthalamidyl moiety.

In view of the foregoing, there is need in the art for new cryptates that have long fluorescence lifetimes and have good emission spectral overlap with the excitation spectrum of acceptors. These features can then be used in assays which provide for an increase in flexibility, reliability and sensitivity in addition to higher throughput. The present invention provides these and other needs.

### BRIEF SUMMARY OF THE INVENTION

In one embodiment, the present invention provides a compound of Formula I:

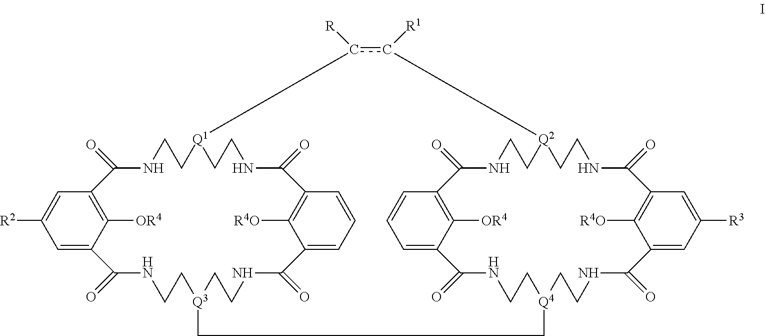

wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and R¹ join to form an optionally substituted cyclopropyl group wherein the dotted bond is absent;
R² and R³ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, or an activated group that can be linked to a biomolecule, wherein the activated group is a member selected from the group consisting of a halogen, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group or L;
R⁴ are each independently a hydrogen, C₁-C₆ alkyl, or alternatively, 3 of the R⁴ groups are absent and the resulting oxides are chelated to a lanthanide cation; and
Q¹-Q⁴ are each independently a member selected from the group of carbon or nitrogen.

In another embodiment, the present invention provides a bioconjugate compound of Formula II:

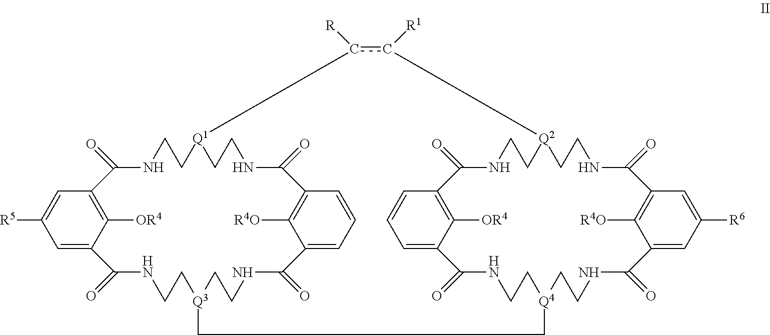

wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and R¹ join to form an optionally substituted cyclopropyl group wherein the dotted bond is absent;
R⁵ and R⁶ are each independently a member selected from the group of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group or -L^{q}-B, wherein -L^{q}-B comprises a linking group and a biomolecule, wherein the compound comprises at least one -L^{q}-B;
R⁴ are each independently a hydrogen, C₁-C₆ alkyl, or alternatively, 3 of the R⁴ groups are absent and the resulting oxides are chelated to a lanthanide cation; and
Q¹-Q⁴ are each independently a member selected from the group of carbon and nitrogen.

In still another embodiment, the present invention provides an assay method for detecting an analyte in solution, the method comprising:
contacting a sample with a compound of Formula II comprising a lanthanide ion, wherein the compound of Formula I binds with an analyte;
exciting the sample with light; and
detecting fluorescence emission to detect the analyte.

These and other aspects, objects and embodiments will become more apparent when read with the detailed description and figures that follow.

### BRIEF DESCRIPTION OF THE DRAWINGS

FIG. 1 illustrates a protein conjugation with a cryptate of the present invention.

FIG. 2 illustrates a protein conjugation with a cryptate of the present invention.

FIG. 3A-E illustrate specific compounds of the present invention.

FIG. 4A-D illustrate specific compounds of the present invention.

FIG. 5 illustrates an excitation band at 365 nm of Lumi4™-Tb³⁺, which produced maximum fluorescence emission at 550 nm.

FIG. 6 illustrates an excitation band at 350-355 nm for alkene cryptate (m) (inventive) complexed with Tb³⁺ (“alkene cryptate (m)-Tb”) for maximum emission at 550 nm.

FIG. 7 illustrates fluorescence emission spectra between 490-620 nm of alkene cryptate (m) and Lumi4™-Tb³⁺ (comparative) at an excitation wavelength of 365 nm.

### DETAILED DESCRIPTION OF THE INVENTION

### I. Definitions

The terms “a,” “an,” or “the” as used herein not only includes aspects with one member, but also includes aspects with more than one member.

The term “about” as used herein to modify a numerical value indicates a defined range around that value. If “X” were the value, “about X” would indicate a value from 0.9X to 1.1X, and more preferably, a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

When the modifier “about” is applied to describe the beginning of a numerical range, it applies to both ends of the range. Thus, “from about 500 to 850 nm” is equivalent to “from about 500 nm to about 850 nm.” When “about” is applied to describe the first value of a set of values, it applies to all values in that set. Thus, “about 580, 700, or 850 nm” is equivalent to “about 580 nm, about 700 nm, or about 850 nm.” However, when the modifier “about” is applied to describe only the end of the range or only a later value in the set of values, it applies only to that value or that end of the range. Thus, the range “about 2 to about 10” is the same as “about 2 to about 10,” but the range “2 to about 10” is not.

“Activated acyl” as used herein includes a —C(O)-LG group. “Leaving group” or “LG” is a group that is susceptible to displacement by a nucleophilic acyl substitution (i.e., a nucleophilic addition to the carbonyl of —C(O)-LG, followed by elimination of the leaving group). Representative leaving groups include halo, cyano, azido, carboxylic acid derivatives such as t-butylcarboxy, and carbonate derivatives such as i-BuOC(O)O—. An activated acyl group may also be an activated ester as defined herein or a carboxylic acid activated by a carbodiimide to form an anhydride (preferentially cyclic) or mixed anhydride —OC(O)R^{a} or —OC(NR^{a})NHR^{b} (preferably cyclic), wherein R^{a} and R^{b} are members independently selected from the group consisting of C₁-C₆ alkyl, C₁-C₆ perfluoroalkyl, C₁-C₆ alkoxy, cyclohexyl, 3-dimethylaminopropyl, or N-morpholinoethyl. Preferred activated acyl groups include activated esters.

“Activated ester” as used herein includes a derivative of a carboxyl group that is more susceptible to displacement by nucleophilic addition and elimination than an ethyl ester group (e.g., an NHS ester, a sulfo-NHS ester, a PAM ester, or a halophenyl ester). Representative carbonyl substituents of activated esters include succinimidyloxy (—OC₄H₄NO₂), sulfosuccinimidyloxy (—OC₄H₃NO₂SO₃H), -1-oxybenzotriazolyl (—OC₆H₄N₃); 4-sulfo-2,3,5,6-tetrafluorophenyl; or an aryloxy group that is optionally substituted one or more times by electron-withdrawing substituents such as nitro, fluoro, chloro, cyano, trifluoromethyl, or combinations thereof (e.g., pentafluorophenyloxy, or 2,3,5,6-tetrafluorophenyloxy). Preferred activated esters include succinimidyloxy, sulfosuccinimidyloxy, and 2,3,5,6-tetrafluorophenyloxy esters.

“Acyl” as used herein includes an alkanoyl, aroyl, heterocycloyl, or heteroaroyl group as defined herein. Representative acyl groups include acetyl, benzoyl, nicotinoyl, and the like.

“Alkanoyl” as used herein includes an alkyl-C(O)— group wherein the alkyl group is as defined herein. Representative alkanoyl groups include acetyl, ethanoyl, and the like.

“Alkenyl” as used herein includes a straight or branched aliphatic hydrocarbon group of 2 to about 15 carbon atoms that contains at least one carbon-carbon double or triple bond. Preferred alkenyl groups have 2 to about 12 carbon atoms. More preferred alkenyl groups contain 2 to about 6 carbon atoms. In one aspect, hydrocarbon groups that contain a carbon-carbon double bond are preferred. In a second aspect, hydrocarbon groups that contain a carbon-carbon triple bond are preferred (i.e., alkynyl). “Lower alkenyl” as used herein includes alkenyl of 2 to about 6 carbon atoms. Representative alkenyl groups include vinyl, allyl, n-butenyl, 2-butenyl, 3-methylbutenyl, n-pentenyl, heptenyl, octenyl, decenyl, propynyl, 2-butynyl, 3-methylbutynyl, n-pentynyl, heptynyl, and the like.

An alkenyl group can be unsubstituted or optionally substituted. When optionally substituted, one or more hydrogen atoms of the alkenyl group (e.g., from 1 to 4, from 1 to 2, or 1) may be replaced with a moiety independently selected from the group of fluoro, hydroxy, alkoxy, amino, alkylamino, acylamino, thio, and alkylthio.

“Alkenylene” as used herein includes a straight or branched bivalent hydrocarbon chain containing at least one carbon-carbon double or triple bond. Preferred alkenylene groups include from 2 to about 12 carbons in the chain, and more preferred alkenylene groups include from 2 to 6 carbons in the chain. In one aspect, hydrocarbon groups that contain a carbon-carbon double bond are preferred. In a second aspect, hydrocarbon groups that contain a carbon-carbon triple bond are preferred. Representative alkenylene groups include —CH═CH—, —CH₂—CH═CH—, —C(CH₃)═CH—, —CH₂CH═CHCH₂—, ethynylene, propynylene, n-butynylene, and the like.

“Alkoxy” as used herein includes an alkyl-O— group wherein the alkyl group is as defined herein. Representative alkoxy groups include methoxy, ethoxy, n-propoxy, i-propoxy, n-butoxy, heptoxy, and the like.

An alkoxy group can be unsubstituted or optionally substituted. When optionally substituted, one or more hydrogen atoms of the alkoxy group (e.g., from 1 to 4, from 1 to 2, or 1) may be replaced with a moiety independently selected from the group of fluoro, hydroxy, alkoxy, amino, alkylamino, acylamino, thio, and alkylthio.

“Alkoxyalkyl” as used herein includes an alkyl-O-alkylene- group wherein alkyl and alkylene are as defined herein. Representative alkoxyalkyl groups include methoxyethyl, ethoxymethyl, n-butoxymethyl and cyclopentylmethyloxyethyl.

“Alkoxycarbonyl” as used herein includes an ester group; i.e., an alkyl-O—CO— group wherein alkyl is as defined herein. Representative alkoxycarbonyl groups include methoxycarbonyl, ethoxycarbonyl, t-butyloxycarbonyl, and the like.

“Alkoxycarbonylalkyl” as used herein includes an alkyl-O—CO-alkylene- group wherein alkyl and alkylene are as defined herein. Representative alkoxycarbonylalkyl include methoxycarbonylmethyl, ethoxycarbonylmethyl, methoxycarbonylethyl, and the like.

“Alkyl” as used herein includes an aliphatic hydrocarbon group, which may be straight or branched-chain, having about 1 to about 20 carbon atoms in the chain. Preferred alkyl groups have 1 to about 12 carbon atoms in the chain. More preferred alkyl groups have 1 to 6 carbon atoms in the chain. “Branched-chain” as used herein includes that one or more lower alkyl groups such as methyl, ethyl or propyl are attached to a linear alkyl chain. “Lower alkyl” as used herein includes 1 to about 6 carbon atoms, preferably 5 or 6 carbon atoms in the chain, which may be straight or branched. Representative alkyl groups include methyl, ethyl, n-propyl, isopropyl, n-butyl, t-butyl, n-pentyl, and 3-pentyl.

An alkyl group can be unsubstituted or optionally substituted. When optionally substituted, one or more hydrogen atoms of the alkyl group (e.g., from 1 to 4, from 1 to 2, or 1) may be replaced with a moiety independently selected from the group of fluoro, hydroxy, alkoxy, amino, alkylamino, acylamino, thio, and alkylthio.

“Alkylene” as used herein includes a straight or branched bivalent hydrocarbon chain of 1 to about 6 carbon atoms. Preferred alkylene groups are the lower alkylene groups having 1 to about 4 carbon atoms. Representative alkylene groups include methylene, ethylene, and the like.

“Alkylthio” as used herein includes an alkyl-S— group wherein the alkyl group is as defined herein. Preferred alkylthio groups are those wherein the alkyl group is lower alkyl.

Representative alkylthio groups include methylthio, ethylthio, isopropylthio, heptylthio, and the like.

“Alkylthioalkyl” as used herein includes an alkylthio-alkylene- group wherein alkylthio and alkylene are defined herein. Representative alkylthioalkyl groups include methylthiomethyl, ethylthiopropyl, isopropylthioethyl, and the like.

“Amido” as used herein includes a group of formula Y₁Y₂N—C(O)— wherein Y₁ and Y₂ are independently hydrogen, alkyl, or alkenyl; or Y₁ and Y₂, together with the nitrogen through which Y₁ and Y₂ are linked, join to form a 4- to 7-membered azaheterocyclyl group (e.g., piperidinyl). Representative amido groups include primary amido (H₂N—C(O)—), methylamido, dimethylamido, diethylamido, and the like. Preferably, “amido” is an —C(O)NRR′ group where R and R′ are members independently selected from the group of H and alkyl. More preferably, at least one of R and R′ is H.

“Amidoalkyl” as used herein includes an amido-alkylene- group wherein amido and alkylene are defined herein. Representative amidoalkyl groups include amidomethyl, amidoethylene, dimethylamidomethyl, and the like.

“Amino” as used herein includes a group of formula Y₁Y₂N— wherein Y₁ and Y₂ are independently hydrogen, acyl, or alkyl; or Y₁ and Y₂, together with the nitrogen through which Y₁ and Y₂ are linked, join to form a 4- to 7-membered azaheterocyclyl group (e.g., piperidinyl). Optionally, when Y₁ and Y₂ are independently hydrogen or alkyl, an additional substituent can be added to the nitrogen, making a quaternary ammonium ion. Representative amino groups include primary amino (H₂N—), methylamino, dimethylamino, diethylamino, and the like. Preferably, “amino” is an —NRR′ group where R and R′ are members independently selected from the group of H and alkyl. Preferably, at least one of R and R′ is H.

“Aminoalkyl” as used herein includes an amino-alkylene- group wherein amino and alkylene are defined herein. Representative aminoalkyl groups include aminomethyl, aminoethyl, dimethylaminomethyl, and the like.

“Aroyl” as used herein includes an aryl-CO— group wherein aryl is defined herein. Representative aroyl include benzoyl, naphth-1-oyl and naphth-2-oyl.

“Aryl” as used herein includes an aromatic monocyclic or multicyclic ring system of 6 to about 14 carbon atoms, preferably of 6 to about 10 carbon atoms. Representative aryl groups include phenyl and naphthyl.

“Aromatic ring” as used herein includes 5-12 membered aromatic monocyclic or fused polycyclic moieties that may include from zero to four heteroatoms selected from the group of oxygen, sulfur, selenium, and nitrogen. Exemplary aromatic rings include benzene, pyrrole, furan, thiophene, imidazole, oxazole, thiazole, triazole, pyrazole, pyridine, pyrazine, pyridazine, pyrimidine, naphthalene, benzathiazoline, benzothiophene, benzofurans, indole, benzindole, quinoline, and the like. The aromatic ring group can be substituted at one or more positions with halo, alkyl, alkoxy, alkoxy carbonyl, haloalkyl, cyano, sulfonato, amino sulfonyl, aryl, sulfonyl, aminocarbonyl, carboxy, acylamino, alkyl sulfonyl, amino and substituted or unsubstituted substituents.

“Biomolecule” as used herein includes a natural or synthetic molecule for use in biological systems. Preferred biomolecules include an antibody, an antibody fragment, an antigen, a protein, a peptide, an enzyme substrate, a hormone, a hapten, an avidin, a streptavidin, a carbohydrate, a carbohydrate derivative, an oligosaccharide, a polysaccharide, and a nucleic acid. More preferred biomolecules include an antibody, a protein, a peptide, an avidin, a streptavidin, or biotin.

“Carboxy” and “carboxyl” as used herein include a HOC(O)— group (i.e., a carboxylic acid) or a salt thereof.

“Carboxyalkyl” as used herein includes a HOC(O)-alkylene- group wherein alkylene is defined herein. Representative carboxyalkyls include carboxymethyl (i.e., HOC(O)CH₂—) and carboxyethyl (i.e., HOC(O)CH₂CH₂—).

“Cycloalkyl” as used herein includes a non-aromatic mono- or multicyclic ring system of about 3 to about 10 carbon atoms, preferably of about 5 to about 10 carbon atoms. More preferred cycloalkyl rings contain 5 or 6 ring atoms. A cycloalkyl group optionally comprises at least one sp²-hybridized carbon (e.g., a ring incorporating an endocyclic or exocyclic olefin). Representative monocyclic cycloalkyl groups include cyclopentyl, cyclohexyl, cyclohexenyl, cycloheptyl, and the like. Representative multicyclic cycloalkyl include 1-decalin, norbornyl, adamantyl, and the like.

“Cycloalkylene” as used herein includes a bivalent cycloalkyl having about 4 to about 8 carbon atoms. Preferred cycloalkylenyl groups include 1,2-, 1,3-, or 1,4-cis- or trans-cyclohexylene.

“Halo” or “halogen” as used herein includes fluoro, chloro, bromo, or iodo.

“Heteroatom” as used herein includes an atom other than carbon or hydrogen. Representative heteroatoms include O, S, and N. The nitrogen or sulphur atom of the heteroatom is optionally oxidized to the corresponding N-oxide, S-oxide (sulfoxide), or S,S-dioxide (sulfone). In a preferred aspect, a heteroatom has at least two bonds to alkylene carbon atoms (e.g., —C₁-C₉ alkylene-O—C₁-C₉ alkylene-). In some embodiments, a heteroatom is further substituted with an acyl, alkyl, aryl, cycloalkyl, heterocyclyl, or heteroaryl group (e.g., —N(Me)-; —N(Ac)—).

“Heteroaroyl” as used herein includes a heteroaryl-C(O)— group wherein heteroaryl is as defined herein. Representative heteroaroyl groups include thiophenoyl, nicotinoyl, pyrrol-2-ylcarbonyl, pyridinoyl, and the like.

“Heterocycloyl” as used herein includes a heterocyclyl-C(O)— group wherein heterocyclyl is as defined herein. Representative heterocycloyl groups include N-methyl prolinoyl, tetrahydrofuranoyl, and the like.

“Hydroxyalkyl” as used herein includes an alkyl group as defined herein substituted with one or more hydroxy groups. Preferred hydroxyalkyls contain lower alkyl. Representative hydroxyalkyl groups include hydroxymethyl and 2-hydroxyethyl.

“Lanthanide” as used herein includes neodymium (Nd), samarium (Sm), europium (Eu), terbium (Tb), dysprosium (Dy) and ytterbium (Yb).

“A water solubilizing group” is a group that imparts more hydrophilicity to the cryptate. A water solubilizing group can be an ethylene oxide oligomer. In certain aspects, water solubilizing groups include one or more alkylene oxide repeat units. For example, a water-solubilizing group can contain one or more ethylene glycol units, —(OCH₂CH₂)ₙ—. The PEG group can be any length, however, typically includes between n is 1 to 20 ethylene glycol repeat units. Other PEG derivatives such as CH₃(OCH₂CH₂)ₙCO or CH₃(OCH₂CH₂)ₙ wherein n=1 to 15, such as 1, 2, 3, 4, 5, 6, 7, 8, 9, 10, 11, 12, 13, 14, or 15, or n is 4 to 8; or HOOCCH₂CH₂CO—, HOOCCH₂CH₂— or CH₃CO— can be used. One of skill in the art will know of other groups that impart more water solubility to the molecule.

### II. Embodiments

A. Cryptates

In one embodiment, the present invention provides a compound of Formula I:

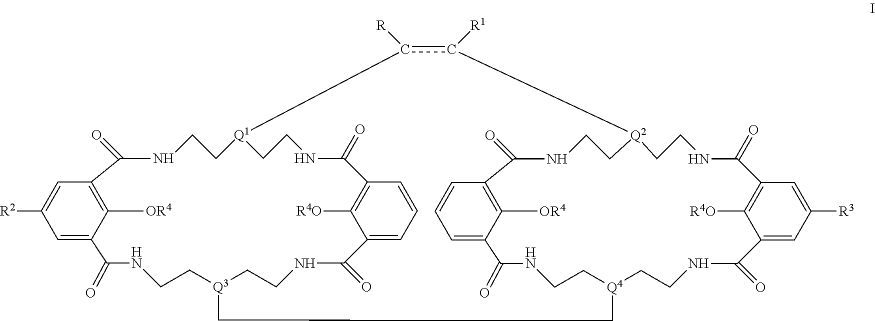

wherein when the dotted line is present, R and R¹ are each independently selected from the group consisting of hydrogen, halogen, hydroxyl, alkyl optionally substituted with one or more halogen (e.g. fluorine) atoms, carboxyl, alkoxycarbonyl, amido, sulfonato, or alkoxycarbonylalkyl or alkylcarbonylalkoxy.

For example, in this abridged formula, the dotted line is present as is shown:

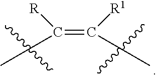

In an alternative embodiment, R and R¹ join to form an optionally substituted cyclopropyl group wherein the dotted bond is absent. In this embodiment, the carbons of the cyclopropyl group fill their valence with hydrogen or an alkyl substituent such as halo, hydroxy, alkoxy, amino, alkylamino, acylamino, thio, and alkylthio. For example, a cyclopropyl group is formed and the carbons can be further substituted as shown with R⁷ and R⁸ being independently selected from the group of hydrogen, halo, hydroxy, alkoxy, amino, alkylamino, acylamino, thio, and alkylthio.

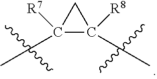

R² and R³ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, or an activated group that can be linked to a biomolecule such as a protein, wherein the activated group is a member selected from the group consisting of a halogen, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group or L;

R⁴ are each independently a hydrogen, C₁-C₆ alkyl, or alternatively, 3 of the R⁴ groups are absent and the resulting oxides are chelated to a lanthanide cation.

Q¹-Q⁴ are each independently a member selected from the group consisting of carbon and nitrogen. In certain instances, Q¹, Q², Q³, and Q⁴ are each carbon. In other instances, Q¹, Q², Q³, and Q⁴ are each nitrogen. Alternatively, Q¹ is carbon and Q² is nitrogen, or Q¹ is nitrogen and Q² is carbon. Alternatively, Q¹ is carbon, Q² is carbon and Q³ and Q⁴ are each nitrogen. Alternatively, Q¹ is carbon, Q² is carbon, Q³ is carbon and Q⁴ is nitrogen. Alternatively, Q¹ is carbon, Q² is carbon, Q³ is nitrogen and Q⁴ is carbon. Alternatively, Q¹ is nitrogen, Q² is nitrogen, Q³ is carbon and Q⁴ is carbon. Alternatively, Q¹ is nitrogen, Q² is carbon, Q³ is carbon and Q⁴ is carbon.

In certain instances, 3 of the R⁴ groups are absent and the resulting oxides are chelated to a lanthanide cation, wherein the cation can be neodymium (Nd), samarium (Sm), europium (Eu), terbium (Tb), dysprosium (Dy) or ytterbium (Yb).

In certain instances, at least one of R² and R³ is L. L is a linkage which is linear or branched, cyclic or heterocyclic, saturated or unsaturated, having 1-60 atoms selected from the group consisting of C, N, P, O, and S, wherein L can have additional hydrogen atoms to fill valences, wherein said linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds. L can be a PEG chain such as CH₃(OCH₂CH₂)ₙ wherein n=1 to 15, such as 1, 2, 3, 4, 5, 6, 7, 8, 9, 10, 11, 12, 13, 14, or 15. In certain instances, L terminates in an activated group.

In certain instances, an activated group is appended to L. For example, L can be —SO₂NR⁹—R¹⁰, COR¹⁰, wherein R⁹ is H, alkyl or aryl and R¹⁰ is alkyl or aryl substituted with the activated group. In certain instances, L terminates in a member selected from the group of an isothiocyanate, an isocyanate, a sulfonylchloride, an aldehyde, a carbodiimide, an acyl azide, an anhydride, a fluorobenzene, a carbonate, a NHS ester, an imidoester, an epoxide or a fluorophenyl ester.

In certain instances, R and R¹ are each hydrogen. In certain instances, at least one of R² and R³ is —SO₂Cl.

In certain instances, at least one of R² and R³ is a member selected from the group of activated acyl, activated ester, optionally substituted alkylsulfonate ester, optionally substituted arylsulfonate ester, optionally substituted amino, formyl, glycidyl, halo, haloacetamidyl, haloalkyl, hydrazinyl, imido ester, isocyanato, isothiocyanato, maleimidyl, mercapto, carboxyl, alkoxycarbonyl, amido, sulfonato, alkoxycarbonylalkyl, or alkoxyalkyl.

In certain instances, at least one of R² and R³ is a member selected from the group of activated acyl, activated ester, optionally substituted alkylsulfonate ester, optionally substituted arylsulfonate ester, formyl, glycidyl, halo, haloacetamidyl, haloalkyl, imido ester, isocyanato, isothiocyanato, or maleimidyl.

In certain instances, at least one of R² and R³ is a member selected from the following group:

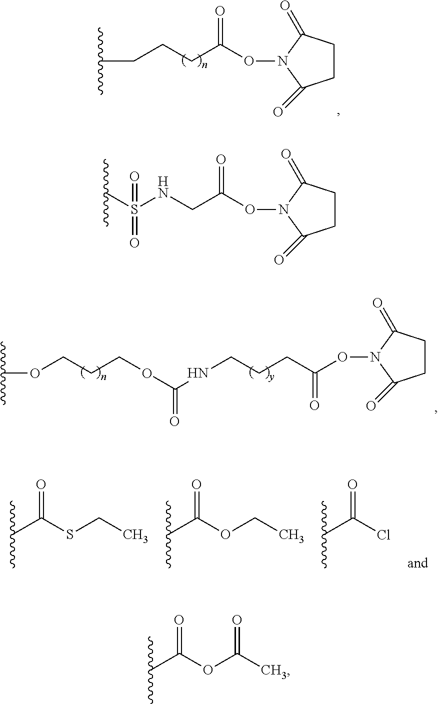

wherein n and y in the above structures are each independently selected from 0 to 5, such as 0, 1, 2, 3, 4, or 5.

In certain instances, a compound of Formula I reacts with a biomolecule (B) to form a bioconjugate of Formula II. In certain instances, the substituents at R² or R³ represent the group before the attachment reaction with B (a biomolecule). In certain instances, “-L^{q}-” comprises the resultant attachment and or linkage between the cryptate of Formula I joined to the biomolecule B.

In certain aspects, the compound of Formula I has a chelated lanthanide cation. For example, in certain instances, the compound of Formula I has the structure:

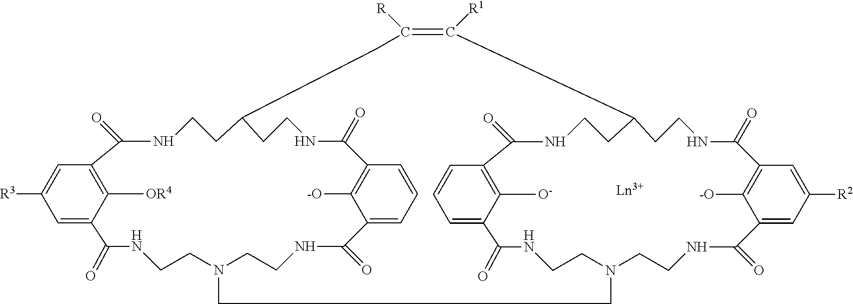

wherein the lanthanide ion is a member selected from the group of neodymium (Nd), samarium (Sm), europium (Eu), terbium (Tb), dysprosium (Dy) or ytterbium (Yb). In certain instances, the lanthanide ion Ln³⁺ is europium or terbium. In certain instances, the lanthanide ion is terbium.

B. Preparation of Compounds of Formula I

Compounds of Formula I are prepared using a multistep synthesis scheme as illustrated in Example I. In brief, 2-methoxyisophthaloyl dichloride is reacted with a solution of thiazolidine-2-thione to generate in good yield (2-methoxy-1,3-phenylene)bis((2-thioxothiazolidin-3-yl)methanone) (Step 1). In Step 2, (2-methoxy-1,3-phenylene)bis((2-thioxothiazolidin-3-yl)methanone) is added dropwise to a solution of N1,N1′-(ethane-1,2-diyl)bis(N₁-(2-aminoethyl)ethane-1,2-diamine) tetrahydrobromide to yield N,N′,N″,N′″-((ethane-1,2-diylbis(azanetriyl))tetrakis(ethane-2,1-diyl))tetrakis(2-methoxy-3-(2-thioxothiazolidine-3-carbonyl)benzamide). This tetra-thioxothiazolidine product is reacted with a tetra ammonium alkene to generate the alkene cryptate in Step 9. The synthesis route however, to the tetra ammonium alkene is itself a 5 step process (See Steps 3-8 in Example I). Briefly, an exocyclic vinyl group is installed onto a cyclohexenyl lactone (Step 3). Step 4 illustrates ring opening of the lactone to generate a diol. The diol is used with a ditert-butyl carbamate bis oxoacetate to generate a dicarbamate moiety in Steps 5 and 6. Metathesis of the dicarbamate generates the protected tetra ammonium alkene in step 7. The tetra ammonium alkene is deprotected in Step 8 and used in Step 9.

The methoxy alkene cryptates can be converted to the phenols as is described in Example II. The phenolic form allows for a chelation to a lanthanide ion or other cation.

### Methods of Labeling Biomolecules

The cryptate compounds of Formula I can be attached to a wide variety of biomolecules. Methods of linking cryptates to various types of biomolecules are known in the art. For a thorough review of, e.g., oligonucleotide labeling procedures, see R. Haugland in Excited States of Biopolymers, Steiner ed., Plenum Press (1983), Fluorogenic Probe Design and Synthesis: A Technical Guide, PE Applied Biosystems (1996), and G. T. Herman, Bioconjugate Techniques, Academic Press (1996).

In certain aspects, the present invention provides a method or process for labeling a ligand or biomolecule with a compound of Formula I, the method comprising: contacting a ligand or biomolecule with a compound having Formula I to generate the corresponding bioconjugate compound of Formula II.

Suitable biomolecule include, but are not limited to, an antibody, an antibody fragment, an antigen, an avidin, a carbohydrate, a deoxy nucleic acid, a dideoxy nucleotide triphosphate, an enzyme cofactor, an enzyme substrate, a fragment of DNA, a fragment of RNA, a hapten, a hormone, a nucleic acid, a nucleotide, a nucleotide triphosphate, a nucleotide phosphate, a nucleotide polyphosphate, an oligosaccharide, a peptide, PNA, a polysaccharide, a protein, a streptavidin, and the like.

In one aspect, the cryptate compounds of Formula I have sufficient solubility in aqueous solutions that once they are conjugated to a soluble ligand or biomolecule, the ligand or biomolecule retains its solubility. In certain instances, the bioconjugates also have good solubility in organic media (e.g., DMSO or DMF), which provides considerable versatility in synthetic approaches to the labeling of desired materials.

In one aspect, the R² or R³ group of the cryptate reacts with a thiol, a hydroxyl, a carboxyl, or an amino group on a biomolecule, forming a linking group between the cryptate (dye) and the biomolecule. In another aspect, this reaction is carried out in mixtures of aqueous buffer and an organic solvent such as DMF at pH 8 to 9. Alternatively, this reaction is carried out in distilled water or in an aqueous buffer solution. For thiols or for acidic groups, a pH of 7 or lower is preferred for the reaction solvent, especially if a substrate also contains a reactive amino group.

Selected examples of reactive functionalities useful for attaching a compound of Formula I to a ligand or biomolecule are shown in Table 1, wherein the bond results from the reaction of a cyptate (dye) with a ligand or biomolecule. Column A of Table 1 is a list of the reactive functionalities, which can be on the compound of Formula I or the biomolecule. Column B is a list of the complementary reactive groups (preferably, a carboxyl, hydroxyl, thiol, or amino functionality), which can be on the biomolecule or the compound of Formula I, and which react with the indicated functionality of Column A to form the bond of Column C. In certain instances, L^{q} comprises the bond of Column C. Those of skill in the art will know of other bonds suitable for use in the present invention.

TABLE 1
Exemplary Bonds for Linking Groups

| A | B | |
|---|---|---|
| Reactive Functionality | Complementary Group | |
| (Compound of | (Biomolecule or | C |
| Formula I or | Compound of | Resulting Linking |
| Biomolecule) | Formula I) | Group |
| activated esters* | amines/anilines | amides |
| acrylamides | thiols | thioethers |
| acyl azides** | amines/anilines | amides |
| acyl halides | amines/anilines | amides |
| acyl halides | alcohols/phenols | esters |
| acyl nitriles | alcohols/phenols | esters |
| acyl nitriles | amines/anilines | amides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| anhydrides | alcohols/phenols | esters |
| anhydrides | amines/anilines | amides/imides |
| aryl halides | thiols | thiophenols |
| aryl halides | amines | aryl amines |
| azides | alkynes | 1,2,3-triazoles |
| azides | ester with phosphine | amide (and phosphine |
| | reagent (e.g., o- | oxide) |
| | diphenylphosphino | |
| | group) | |
| aziridines | thiols | thioethers |
| boronates | glycols | boronate esters |
| boronates/boronic acids | aryl halides | C—C bond to aryl ring |
| boronates/boronic acids | alkenyl halides | C—C bond to alkenyl |
| | | group |
| activated carboxylic | amines/anilines | amides |
| acids | | |
| activated carboxylic | alcohols | esters |
| acids | | |
| activated carboxylic | hydrazines | hydrazides |
| acids | | |
| carbodiimides | carboxylic acids | N-acylureas or |
| | | anhydrides |
| diazoalkanes | carboxylic acids | esters |
| electron-rich diene | dienophile (e.g., | cyclohexene (Diels- |
| | electron-poor alkene) | Alder cycloaddition) |
| epoxides | thiols | thioethers |
| epoxides | amines | alkyl amines |
| epoxides | carboxylic acids | esters |
| haloacetamides | thiols | thioethers |
| haloplatinate | amino | platinum complex |
| haloplatinate | heterocycle | platinum complex |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| photoactivatable | varies; see definition | varies; see definition |
| group | | |
| quadricyclanes | π-electrophile (e.g., | norbornene |
| | Ni bis(dithiolene)) | cycloaddition product |
| silyl halides | alcohols | silyl ethers |
| sulfonyl azides | thiocarboxylic acids | N-acyl sulfonamides |
| sulfonate esters | amines/anilines | alkyl amines |
| sulfonate esters | carboxylic acids | esters |
| sulfonate esters | thiols | thioethers |
| sulfonate esters | alcohols/phenols | ethers |
| sulfonyl halides | amines/anilines | sulfonamides |
| 1,2,4,5-tetrazine | alkene | dihydropyradazine |
| vinyl sulfonyl | thiols | thioethers |
| vinyl sulfonyl | activated diene | cyclohexenyl (Diels- |
| | | Alder) |

*Activated esters, as understood in the art, generally have the formula —C(O)OM, where —OM is a leaving group (e.g. succinimidyloxy (—OC₄H₄NO₂), sulfosuccinimidyloxy (—OC₄H₃NO₂SO₃H), -1-oxybenzotriazolyl (—OC₆H₄N₃); 4-sulfo-2,3,5,6-tetrafluorophenyl; or an aryloxy group or aryloxy substituted one or more times by electron withdrawing substituents such as nitro, fluoro, chloro, cyano, or trifluoromethyl, or combinations thereof, used to form activated aryl esters; or —C(O)OM is a carboxylic acid activated by a carbodiimide to form an anhydride or mixed anhydride —C(O)OC(O)R^{a} or —C(O)OC(NR^{a})NHR^{b}, wherein R^{a} and R^{b} are members independently selected from the group consisting of C₁-C₆ alkyl, C₁-C₆ perfluoroalkyl, C₁-C₆ alkoxy, cyclohexyl, 3-dimethylaminopropyl, or N-morpholinoethyl).
**Acyl azides can also rearrange to isocyanates.

When linking a compound of Formula I having a carboxylic acid with an amine-containing ligand or biomolecule, the carboxylic acid can first be converted to a more reactive form, e.g, a N-hydroxy succinimide (NHS) ester or a mixed anhydride, by means of an activating reagent. The amine-containing ligand or biomolecule is treated with the resulting activated acyl to form an amide linkage. In certain aspects, this reaction is carried out in aqueous buffer at pH 8 to 9 with DMSO or DMF as an optional co-solvent. Alternatively, this reaction is carried out in distilled water or in an aqueous buffer solution.

Similarly, the attachment of an isocyanate- or isothiocyanate-containing compound of Formula I is analogous to the procedure for the carboxy dye, but no activation step is required. The amine-containing ligand or biomolecule is treated directly with the activated acyl compound to form a urea or a thiourea linkage. In a more preferred embodiment, the reaction is carried out in aqueous buffer at pH 9 to 10 with DMSO or DMF as an optional co-solvent. Alternatively, this reaction is carried out in distilled water or in an aqueous buffer solution.

In another aspect, the compound of Formula I has a carboxylic acid and is reacted with a EDC crosslinker to form a o-acylisourea intermediate. This intermediate can react with a biomolecule having a primary amine to form an amide bioconjugate. Alternatively, the o-acylisourea intermediate can be reacted with an amine-reactive sulfo-NHS ester. The sulfo-NHS ester can be reacted with a biomolecule with a primary amine to form an amide bioconjugate.

In one aspect, the biomolecule is an antibody. It is preferred that antibody labeling is carried out in a buffer optionally including an organic co-solvent, under basic pH conditions, and at room temperature. It is also preferred that the labeled antibody be purified by dialysis or by gel permeation chromatography using equipment such as a SEPHADEX® G-50 column to remove any unconjugated compound of Formula I. Those of skill in the art will know of other ways and means for purification.

In still another aspect, the biomolecule contains a thiol group that forms the linking group by reaction with a maleimidyl substituent at R² or R³. In one aspect, the biomolecule is a protein, a peptide, an antibody, a thiolated nucleotide, or a thiolated deoxynucleotide.

In one aspect, biomolecules can be labeled according to the present invention by means of a kit. In certain instances, the kit comprises a buffer and a dye as disclosed in the instant application (i.e., a compound of Formula I). Preferably, the kit contains a coupling buffer such as 1 M KH₂PO₄ (pH 5), optionally with added acid or base to modify the pH (e.g., pH 8.5 is preferred for reactions with succinimide esters and pH 7 is preferred for reactions with maleimides). Preferably, the buffer has a qualified low fluorescence background.

Optionally, the kit can contain a purification sub-kit. After labeling a biomolecule with a preferred dye, the labeled biomolecule may be separated from any side reaction products and any free hydrolyzed product resulting from normal hydrolysis. For biomolecules containing 13 or fewer amino acids, preparative thin layer chromatography (TLC) can remove impurities. In certain instances, preparative TLC, optionally performed with commercially available TLC kits, can be used to purify dye-labeled peptides or proteins.

For larger biomolecules such as larger peptides or proteins, a SEPHADEX® G-15, G-25, or G-50 resin may remove unwanted derivatives. In certain instances, a Gel Filtration of Proteins Kit, which is commercially available from Life Sciences (or GE Healthcare Bio-Sciences, Marborough, Mass.), can be used to separate dye-labeled peptides and proteins from free dye. The labeled biomolecules that remain after desalting can often be used successfully without further purification. In some cases, it may be necessary to resolve and assess the activity of the different products by means of HPLC or other chromatographic techniques.

C. Bioconjugate Compounds

In another embodiment of the invention, a bioconjugate of the Formula II is provided:

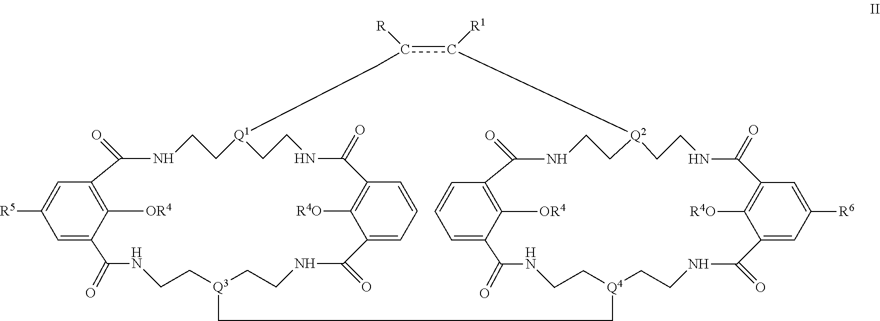

wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy.

In an alternative embodiment, R and R¹ join to form an optionally substituted cyclopropyl group wherein the dotted bond is absent.

R⁵ and R⁶ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl or -L^{q}-B, wherein -L^{q}-B comprises a linking group and a biomolecule, wherein the compound comprises at least one -L^{q}-B;

-L^{q}-B comprises a linking group and a biomolecule, wherein the compound comprises at least one -L^{q}-B. B is a biomolecule. L^{q} comprises the resultant bond between the cryptate and the biomolecule.

R⁴ are each independently a hydrogen, C₁-C₆ alkyl, or alternatively, 3 of the R⁴ groups are absent and the resulting oxides are chelated to a lanthanide cation.

Q¹-Q⁴ are each independently a member selected from the group of carbon and nitrogen. In certain instances, Q¹, Q², Q³, and Q⁴ are each carbon. In other instances, Q¹, Q², Q³, and Q⁴ are each nitrogen. Alternatively, Q¹ is carbon and Q² is nitrogen, or Q¹ is nitrogen and Q² is carbon. Alternatively, Q¹ is carbon, Q² is carbon and Q³ and Q⁴ are each nitrogen. Alternatively, Q¹ is carbon, Q² is carbon, Q³ is carbon and Q⁴ is nitrogen. Alternatively, Q¹ is carbon, Q² is carbon, Q³ is nitrogen and Q⁴ is carbon. Alternatively, Q¹ is nitrogen, Q² is nitrogen, Q³ is carbon and Q⁴ is carbon. Alternatively, Q¹ is nitrogen, Q² is carbon, Q³ is carbon and Q⁴ is carbon.

In certain aspects, a biomolecule for the instant invention is selected from the group containing an antibody, an antigen, an avidin, a carbohydrate, a deoxy nucleic acid, an enzyme cofactor, an enzyme substrate, a fragment of DNA, a fragment of RNA, a hapten, a hormone, a nucleic acid, a nucleotide, a nucleotide triphosphate, a nucleotide phosphate, a nucleotide polyphosphate, an oligosaccharide, a peptide, PNA, a polysaccharide, a protein, a streptavidin, and the like.

In certain instances, B is an antibody, antibody fragment, protein or peptide.

In certain instances, L^{q} comprises a resultant bond such as an amide, ether, thioether, ester, thioester, carbamate, urea, or thiourea. L^{q} optionally comprises any additional atoms making up the linkage between the cryptate and the biomolecule.

In certain instances, the bioconjugate of Formula II has the following structure:

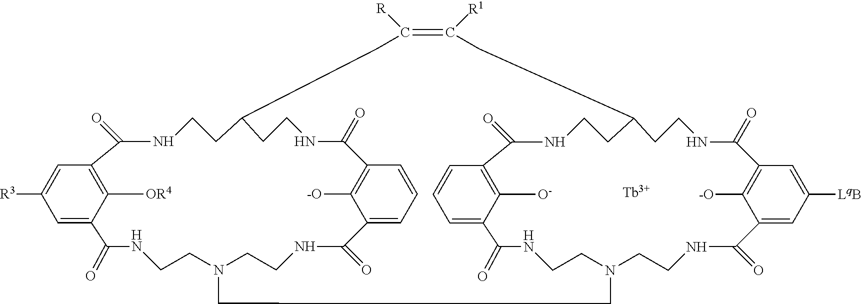

In certain instances, B is an antibody or an antibody fragment.

D. Cryptates of Formula III

In certain other aspects, the present invention provides a compound of Formula III:

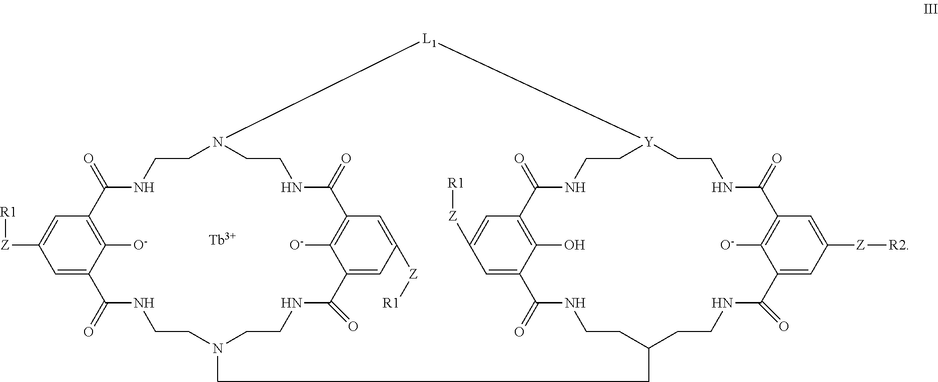

In certain aspects, Y in Formula III is CH or N; and L₁ is ˜CH₂CH₂˜ or when Y is CH, L₁ can be ˜CH═CH˜.

R¹ is hydrogen or a water solubilizing group such as CH₃(OCH₂CH₂)ₙCO or CH₃(OCH₂CH₂)ₙ wherein n=1 to 15, such as 1, 2, 3, 4, 5, 6, 7, 8, 9, 10, 11, 12, 13, 14, or 15, or n is 4 to 8, or R¹ can be HOOCCH₂CH₂CO—, HOOCCH₂CH₂— or CH₃CO—.

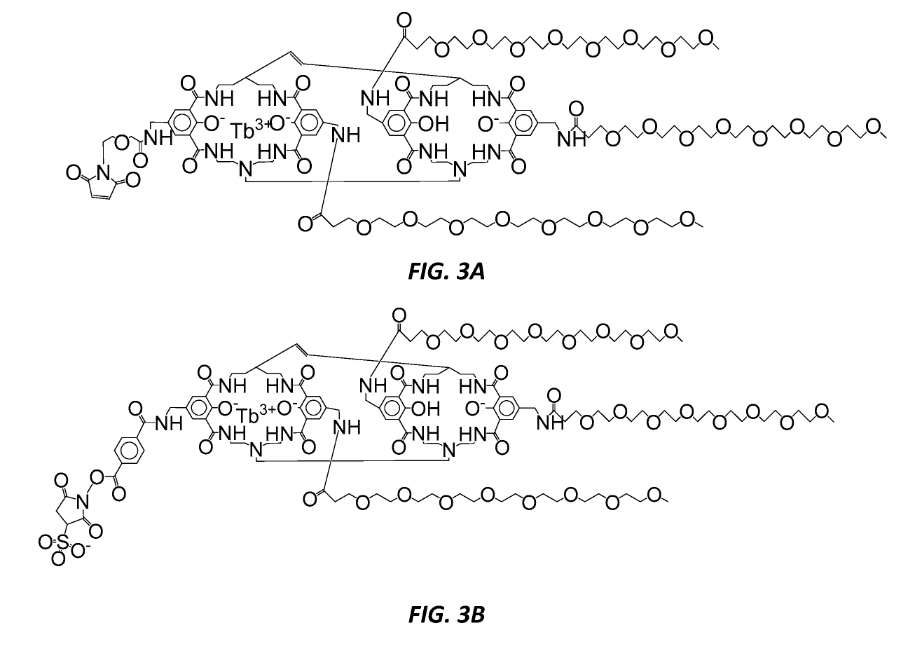

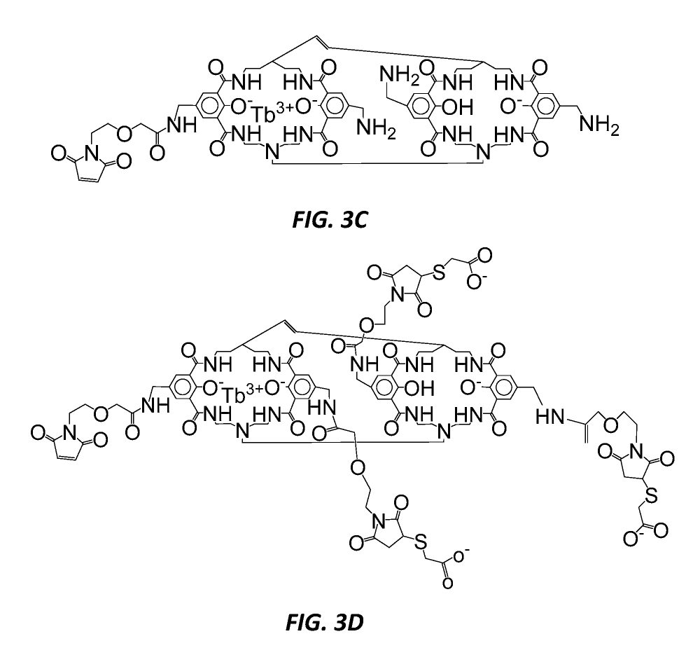

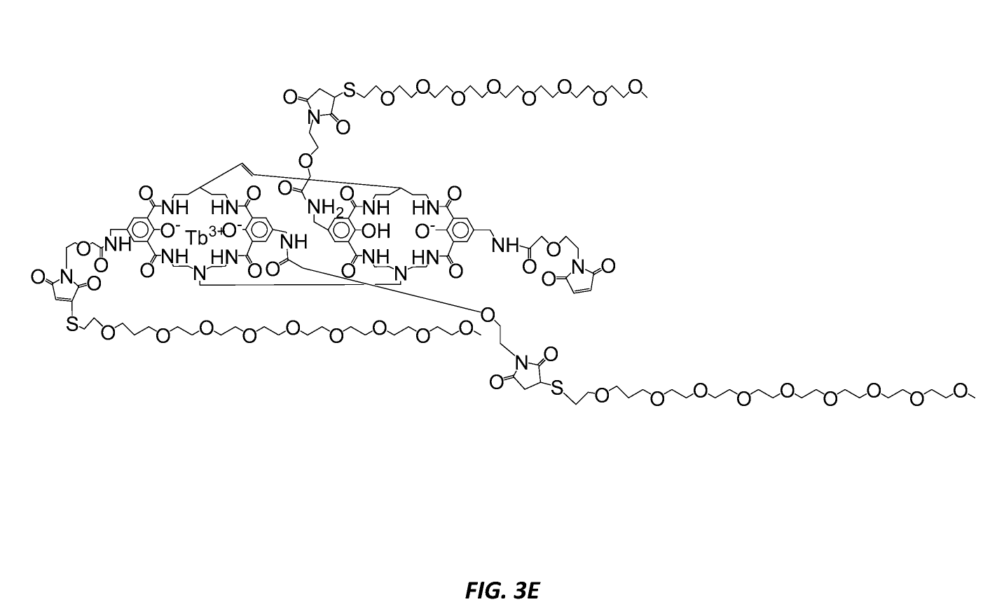

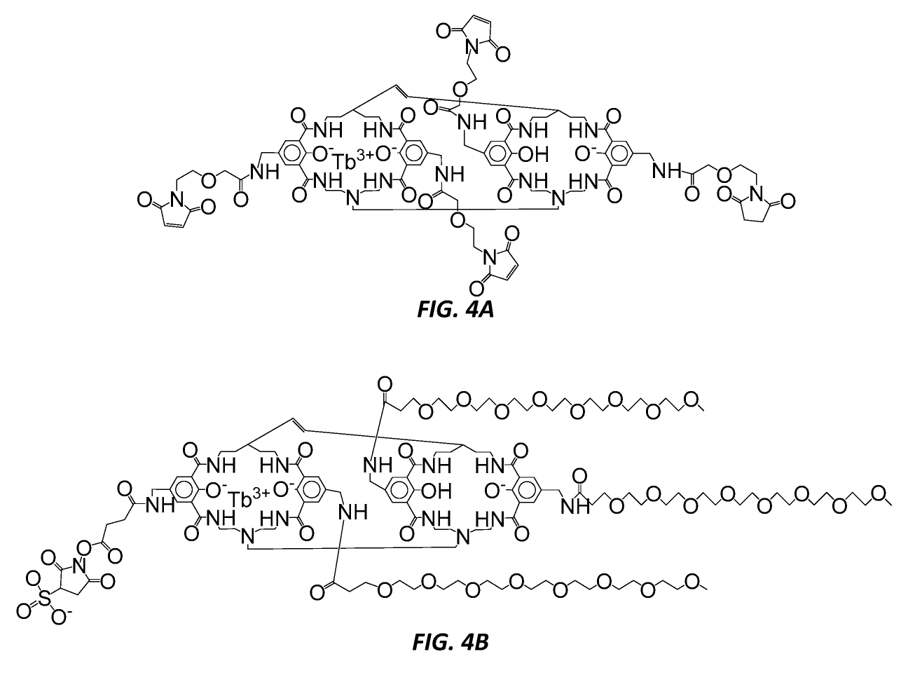

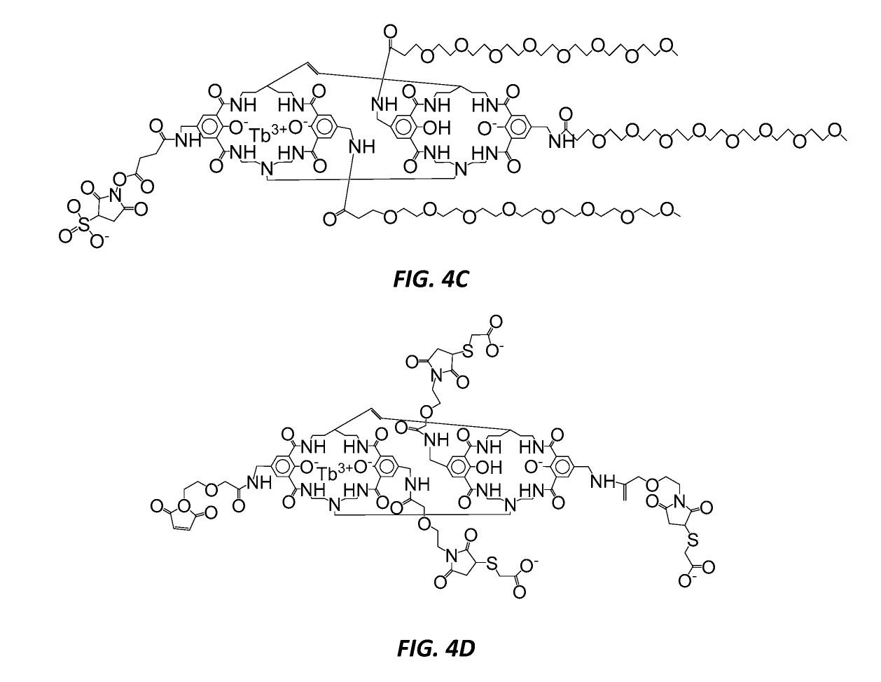

R¹ can optionally also be formed from a reactive group R² (as defined below) and a water soluble thiol (when R² comprises a maleimide) or amine (when R² contains an activated ester, thiocyanate, cyanate, SO₂C₁, or SO₂F). For example, see specific compounds in FIGS. 3 and 4.

R² is a protein reactive group such as maleimide, NHS ester, sulfo-NHS ester, pentafluorophenyl ester, tetrafluorophenyl ester, SO₂C₁, or SO₂F.

Z is a bifunctional link such as —NR³(CH₂)ₙ wherein n is 0 to 10, such as 0, 1, 2, 3, 4, 5, 6, 7, 8, 9, 10, but preferably n is 1 to 3, or —NR³—(CH₂CH₂O)ₙ(CH₂)ₘ where m is 0 to 3 such as 0, 1, 2, or 3 and n is 1 to 5, such as 1, 2, 3, 4, 5, but preferably n is 1 to 3 and n is 1.

R³ is hydrogen, C₁-C₅ alkyl (C₁ C₂ C₃ C₄ or C₅) or methoxyethyl, but preferably, R³ is hydrogen.

In other aspects, Z—R² can be —N═C═S, N═C═O, —CH₂N═C═S, or —CH₂N═C═O.

In certain other aspects, Y is CH or N and L₁ is ˜CH₂CH₂˜ or when Y is CH, L₁ can be ˜CH═CH˜.

In certain aspects, R¹ can be the same as R² which can be protein reactive group such as maleimide, NHS ester, sulfo-NHS ester, pentafluorophenyl ester, tetrafluorophenyl ester, SO₂C₁, or SO₂F.

Z is bifunctional link such as —NR³(CH₂)ₙ wherein n=0 to 10, such as 1, 2, 3, 4, 5, 6, 7, 8, 9, or 10 but preferably n is 1 to 3, or —NR³—(CH₂CH₂O)ₙ(CH₂)ₘ where m is 0 to 3 such as 0, 1, 2, or 3, and n is 1 to 5 such as 1, 2, 3, 4, 5, but preferably n is 1 to 3 and n is 1.

R³ is hydrogen, C₁-C₅ alkyl, or methoxyethyl, but preferably R³ is hydrogen.

In certain aspects, Z—R² can be —N═C═S, N═C═O, CH₂N═C═S, or CH₂N═C═O.

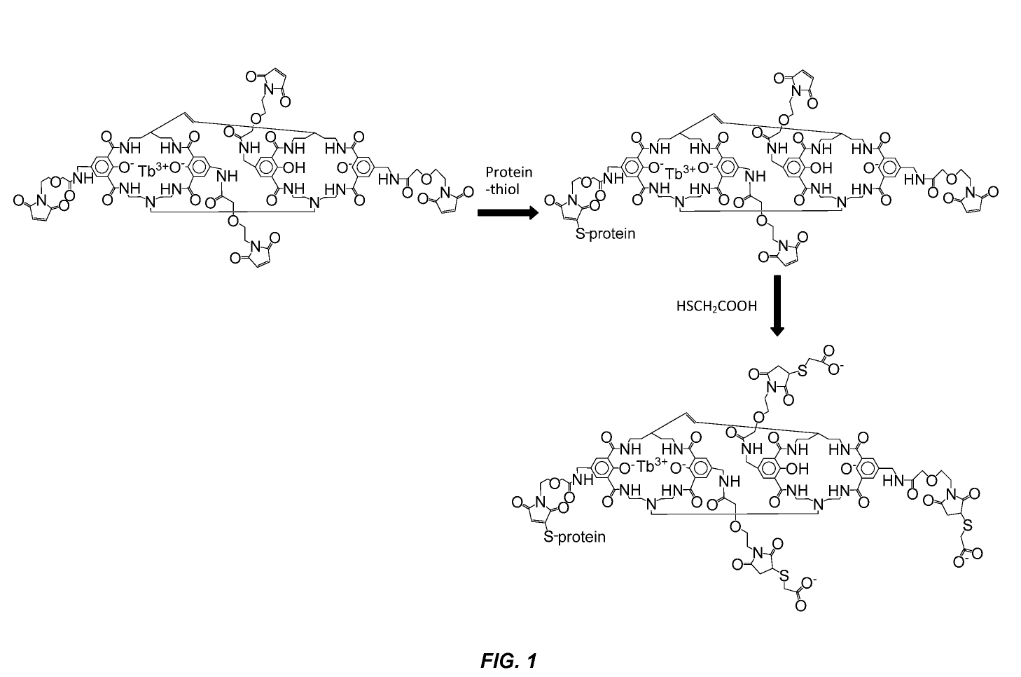

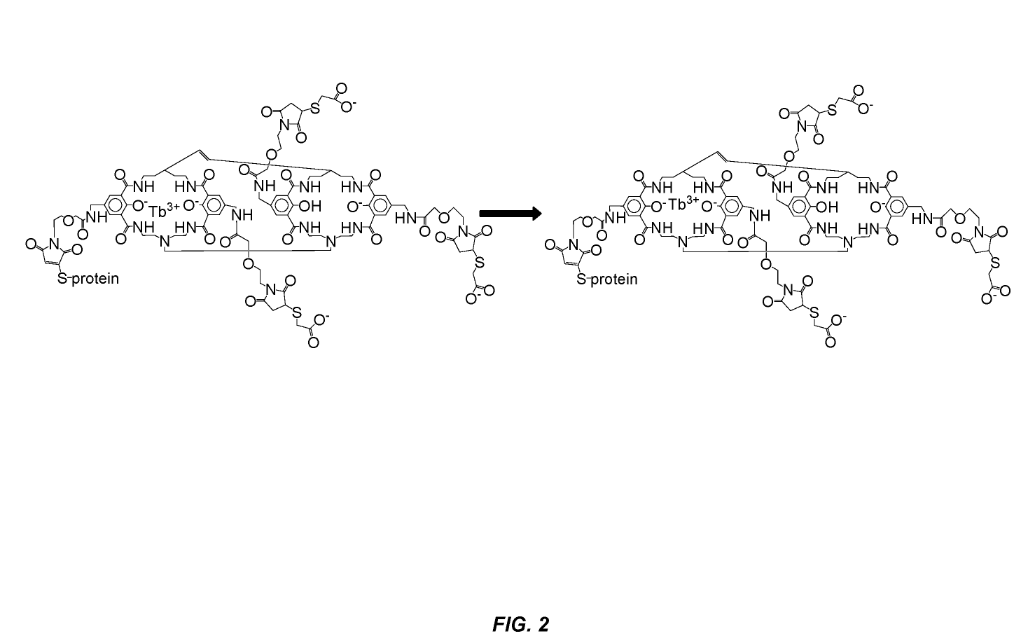

In certain aspects, compounds of Formula III, which may be tetrafunctionalized, can be linked to a biomolecule. For example, these tetrafunctionalized molecules are linked to a protein such as an antibody, through one of the 4 functionalized reactive groups, then treated with excess water and a soluble thiol (for a maleimide reactive group) or amine (for ester or thiocyanate reactive group) to inactivate the other 3 reactive groups. For example, if the thiol-containing protein is treated with the above compound, followed by mercaptoacetic acid, the resulting protein cryptate complex is shown in FIG. 1. FIG. 2 shows another compound of Formula III attached to a protein.

In certain aspects, compounds of Formula III can be a sub-generic formula of compounds of Formula I. In other words, Q¹-Q⁴ of Formula I are the following in Formula III: Q¹ and Q³ are both nitrogen; Q⁴ is carbon and Q² can be nitrogen or carbon.

In certain aspects, the abridged formula below of Formula I is equivalent to L₁ of Formula III.

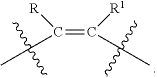

In certain other aspects, R² and R³ of Formula I are equivalent to ZR¹ and ZR³ of Formula III.

In still other aspects, specific compounds of Formula III are shown in FIGS. 3 and 4.

A skilled artisan will appreciate that the compounds of Formula III can be synthesized in similar fashion to compounds of Formula I.

E. Methods of Use

A detectable optical response as used herein includes a change in, or occurrence of, an optical signal that is detectable either by observation or instrumentally. Typically, the detectable response is a change in light or fluorescence, such as a change in the intensity, excitation or emission wavelength distribution of fluorescence, fluorescence lifetime, fluorescence polarization, or a combination thereof. The degree and/or location of staining, compared with a standard or expected response, indicates whether and to what degree the sample possesses a given characteristic. Some compounds of the invention may exhibit little fluorescence emission, but are still useful as quenchers or chromophore dyes. Such chromophores are useful as energy acceptors in FRET applications, or to simply impart the desired color to a sample or portion of a sample.

FRET is a process by which a donor molecule (e.g., a cryptate dye) absorbs light, entering an excited state. Rather than emitting light, the first molecule transfers its excited state to an acceptor molecule with other properties (e.g., a dye fluorescing at a different wavelength or a quencher), and the acceptor fluoresces or quenches the excitation. Because the efficiency of the transfer is dependent on the two molecules' proximity, it can indicate information about molecular complex formation or biomolecular structure.

### III. Examples

Example I illustrates a multistep synthesis scheme including steps A-H to yield compounds of the present invention.

### A. Step 1

### Synthesis of (2-methoxy-1,3-phenylene)bis((2-thioxothiazolidin-3-yl)methanone) (c)

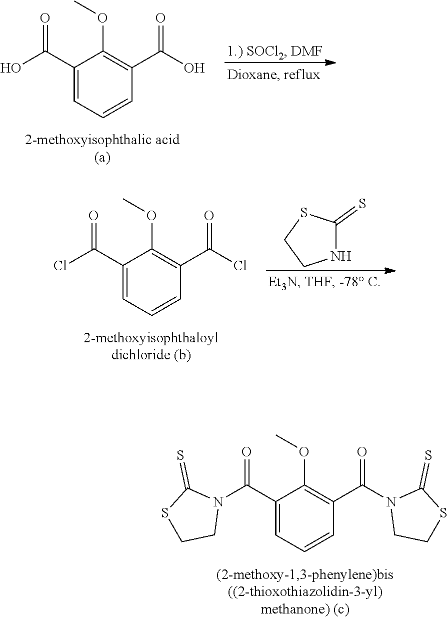 [SMILES: COc1c(C(=O)O)cccc1C(=O)O ; COc1c(C(=O)Cl)cccc1C(=O)Cl ; S=C1NCCS1 ; COc1c(C(=O)N2CCSC2=S)cccc1C(=O)N1CCSC1=S]

To a stirred suspension of 2-methoxyisophthalic acid (a) (5.00 g, 25.5 mmol) in 50 ml of 1,4-dioxane was carefully added thionyl chloride (6.5 ml, 89.3 mmol) and 50 μl of anhydrous dimethylformamide. The reaction was heated, and refluxed for 16 hours under an inert atmosphere. Once cooled, the reaction was concentrated by rotary evaporation which had been flushed with argon, and immediately dried under high vacuum for 16 hours. The crude acid chloride (b) was dissolved in 62 ml of anhydrous dichloromethane and cooled to −78° C. A solution of thiazolidine-2-thione (6.70 g, 56.1 mmol) and triethylamine (7.0 ml, 50.2 mmol) dissolved in 85 ml of anhydrous dichloromethane was added dropwise at that temperature, and the reaction was stirred at room temperature for 16 hours under an inert atmosphere. The bright yellow reaction mixture was washed with brine, 10% hydrochloric acid (aq.), and twice with 10% sodium hydroxide (aq.). The combined organics were dried over sodium sulfate and concentrated in vacuo. The crude material was purified via flash chromatography (silica gel, 120 g, 15-70% ethyl acetate/hexane) to provide (2-methoxy-1,3-phenylene)bis((2-thioxothiazolidin-3-yl)methanone) (c) (6.56 g, 77% yield) as a yellow powder. ¹H NMR (499 MHz, Chloroform-d) δ 7.43 (d, J=7.6 Hz, 2H), 7.13 (t, J=7.7 Hz, 1H), 4.59 (t, J=7.3 Hz, 4H), 3.90 (s, 3H), 3.41 (t, J=7.3 Hz, 4H); MS: mass calculated for C₁₅H₁₄N₂O₃S₄: 397.99; found: positive: 399.7 (M+H)⁺, 421.6 (M+Na)⁺.

### B. Step 2

### Synthesis of N,N′,N″,N′″-((ethane-1,2-diylbis(azanetriyl))tetrakis(ethane-2,1-diyl))tetrakis(2-methoxy-3-(2-thioxothiazolidine-3-carbonyl)benzamide) (e)

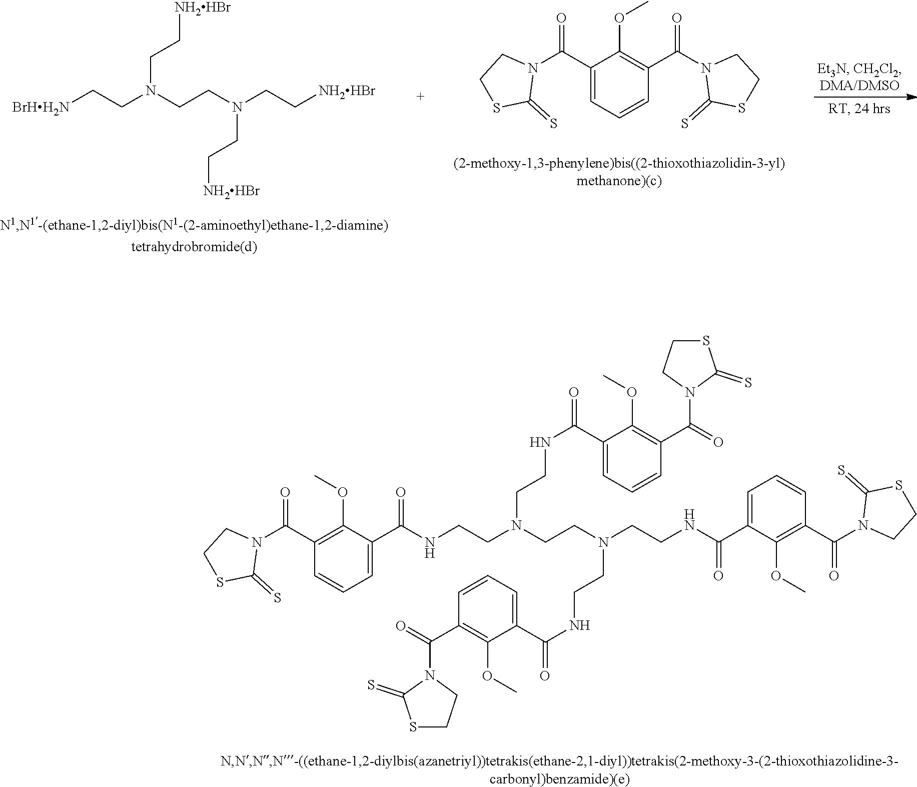

To a solution of (2-methoxy-1,3-phenylene)bis((2-thioxothiazolidin-3-yl)methanone) (e, 4.0 g, 10.0 mmol) in CH₂C₁₂ (80 ml) was added dropwise a solution of N1,N1′-(ethane-1,2-diyl)bis(N1-(2-aminoethyl)ethane-1,2-diamine) tetrahydrobromide (d, 211 mg, 0.38 mmol) and trimethylamine (0.80 ml) dissolved in 6 ml DMA and 2 ml DMSO with syringe pump at a rate of 1 ml/hour. The reaction mixture was stirred under argon for two days before being diluted with 120 ml DCM, and the DCM solution was washed with water (100 ml×3). The organic layer was dried with sodium sulfate and the solvent removed. The resulting residue was directly purified by column chromatography (40 g, silica gel column, 0-25% isopropanol/DCM) to provide N,N′,N″,N′″-((ethane-1,2-diylbis(azanetriyl))tetrakis(ethane-2,1-diyl))tetrakis(2-methoxy-3-(2-thioxothiazolidine-3-carbonyl)benzamide) (e) (180 mg, 33% yield) as a yellow powder. ¹H NMR (499 MHz, Chloroform-d) δ 8.02 (d, J=7.8 Hz, 4H), 7.73 (s, 4H), 7.40 (dd, J=7.6, 1.8 Hz, 4H), 7.18 (t, J=7.6 Hz, 4H), 4.64 (t, J=7.3 Hz, 8H), 3.86 (s, 12H), 3.80 (s, 4H), 3.53 (s, 4H), 3.43 (t, J=7.3 Hz, 8H), 2.74 (d, J=29.8 Hz, 8H), 2.61 (s, 4H); MS: mass calculated for C₅₈H₆₄N₁₀O₁₂S₈: 1348.3; found: positive: 1349.9 (M+H)⁺.

### C. Step 3

### Synthesis of 4-vinyltetrahydro-2H-pyran-2-one (g)

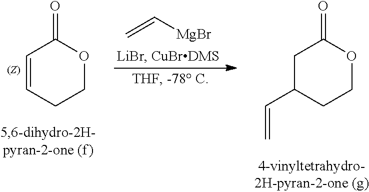

A −78° C. solution of lithium bromide (2.66 g, 30.6 mmol) and copper bromide dimethyl sulfide complex (3.15 g, 15.3 mmol) suspended in 35 ml of anhydrous tetrahydrofuran was added vinyl magnesium bromide (1.0 M, 30.5 ml) dropwise via syringe pump. Once the addition was complete, the reaction was stirred for an additional 30 minutes at that temperature. To the solution was added 5,6-dihydro-2H-pyran-2-one (f) (0.50 g, 5.10 mmol) dissolved in 10 ml of anhydrous tetrahydrofuran dropwise via syringe pump and was stirred for 20 minutes at −78° C. The reaction was warmed to −40° C. and stirred for 1 hour before being quenched with saturated ammonium chloride. The reaction was warmed to room temperature and solids were removed by filtration washing multiple times with diethyl ether. The combined filtrates were washed with brine, dried over sodium sulfate, and concentrated in vacuo. The crude material was purified via flash chromatography (silica gel, 12 g, 0-50% ethyl acetate/hexane) to provide 4-vinyltetrahydro-2H-pyran-2-one (g) (0.408 g, 64% yield) as a pale yellow oil. ¹H NMR (500 MHz, Chloroform-d) δ 5.86-5.70 (m, 1H), 5.17-5.04 (m, 2H), 4.43 (dt, J=11.4, 4.7 Hz, 1H), 4.30 (ddd, J=11.4, 9.9, 3.9 Hz, 1H), 2.79-2.61 (m, 2H), 2.45-2.31 (m, 1H), 2.06-1.94 (m, 1H), 1.72 (dtd, J=14.4, 9.8, 4.8 Hz, 1H); MS: mass calculated for C₇H₁₀O₂: 126.07; found: positive: 127.2 (M+H)⁺, 149.2 (M+Na)⁺.

### D. Step 4

### Synthesis of 3-vinylpentane-1,5-diol (h)

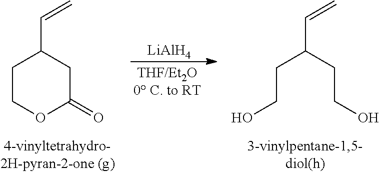

Procedure: A solution of 4-vinyltetrahydro-2H-pyran-2-one (g, 0.93 g, 7.49 mmol) in THF (30 ml) was added slowly to a 0° C. slurry of lithium aluminum hydride (1.10 g, 29.96 mmol) in Et₂O (30 ml), once the addition was complete the reaction mixture was slowly warmed up to RT and stirred for 16 h. A small work-up, followed by TLC indicates that the starting material is completely consumed. The reaction was cooled to 0° C. and 15% NaOH solution (3 ml) was added dropwise to quench the reaction, followed by 2 ml of water. The reaction slurry was diluted with 30 ml diethyl ether and then filtered through a plug of silica gel, and the solid was washed with diethyl ether. The organic phases were combined, solvent removed under vacuo, and the crude product was purified by column chromatography (24 g, silica gel column, 40-100% EtOAc/hexane) to provide 3-vinylpentane-1,5-diol (h, 0.6 g, 61.5% yield) as colorless oil. ¹H NMR (500 MHz, Chloroform-d) δ 5.60 (ddd, J=17.1, 10.1, 9.0 Hz, 1H), 5.15-4.95 (m, 2H), 3.75-3.51 (m, 4H), 2.36 (qt, J=9.3, 4.8 Hz, 1H), 2.02 (d, J=3.9 Hz, 2H), 1.68 (dddd, J=13.8, 7.4, 6.4, 4.8 Hz, 2H), 1.54 (ddt, J=13.7, 9.3, 5.9 Hz, 2H); MS: mass calculated for C₇H₁₄O₂: 130.1; found: positive: 153.1 (M+Na)⁺.

### E. Step 5 & 6

### Synthesis of di-tert-butyl (3-vinylpentane-1,5-diyl)dicarbamate (j)

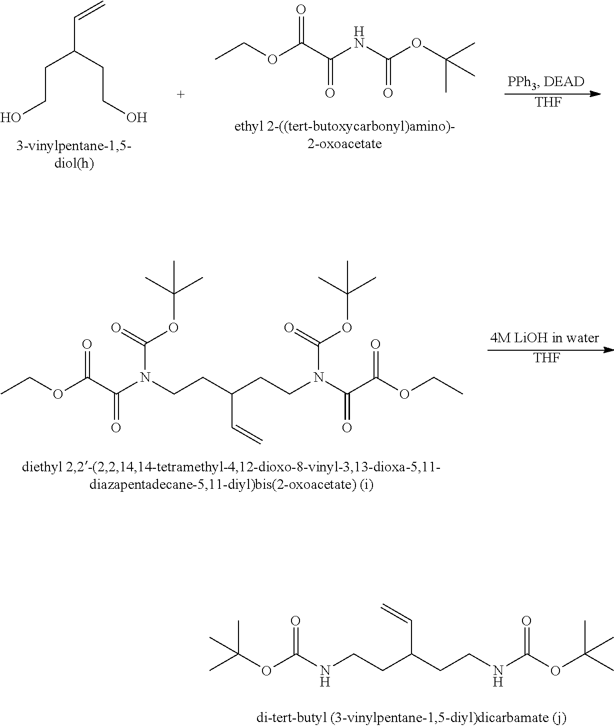

A solution of DEAD (2.39 g, 13.74 mmol) in THF (18 ml) was added dropwise to a solution of 3-vinylpentane-1,5-diol (h, 1.05 g, 8.07 mmol), ethyl 2-((tert-butoxycarbonyl)amino)-2-oxoacetate (4.41 g, 20.3 mmol), and triphenylphosphine (5.33 g, 20.3 mmol) dissolved in THF (30 ml) at 0° C. After completion, the reaction was warmed up to RT and stirred for 16 hours. The solvent was removed under vacuo, the resulting residue dissolved in 50 ml of THF, and 10 ml of 4M LiOH (aq.) solution was added slowly at 0° C. After complete addition, the reaction was warmed up to RT and stirred for 16 hours. Solvent was removed under vacuo, and the residue was dissolved in 150 ml EtOAc and 100 ml water. The organic layer was separated, and the aqueous solution was extracted with EtOAc (100 ml×2). The organic layers were combined and dried with sodium sulfate. After removing solvent, the residue was purified by column chromatography (80 g, silica gel column, 0-40% EtOAc/hexane) to provide di-tert-butyl (3-vinylpentane-1,5-diyl)dicarbamate (j) (1.30 g, 49% yield). ¹H NMR (499 MHz, Chloroform-d) δ 5.54 (ddd, J=17.0, 10.3, 8.9 Hz, 1H), 5.08-4.99 (m, 2H), 4.53 (s, 2H), 3.15 (s, 2H), 3.05 (s, 2H), 2.08 (qt, J=9.2, 4.7 Hz, 1H), 1.62-1.53 (m, 4H), 1.44 (s, 18H); MS: mass calculated for C₁₇H₃₂N₂O₄: 328.24; found: positive: 329.5 (M+H)⁺, 351.4 (M+Na)⁺.

### F. Step 7

### Synthesis of di-tert-butyl (3,6-bis(2-((tert-butoxycarbonyl)amino)ethyl)oct-4-ene-1,8-diyl)(E)-dicarbamate (k)

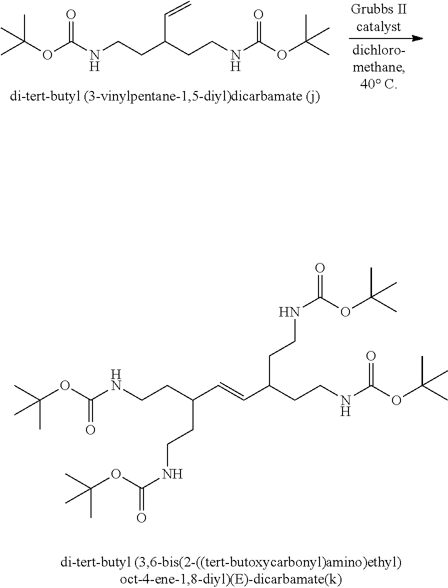

To a solution of di-tert-butyl (3-vinylpentane-1,5-diyl)dicarbamate (j) (0.770 g, 2.34 mmol) dissolved in 19 ml of degassed dichloromethane was added Grubbs 2^{nd} generation catalyst (0.200 g, 0.234 mmol) and the reaction was stirred at reflux for 48 hours under an inert atmosphere. The crude material was filtered through a pad of celite and directly purified by flash chromatography (silica gel, 40 g, 10-60% ethyl acetate/hexane) to provide di-tert-butyl (3,6-bis(2-((tert-butoxycarbonyl)amino)ethyl)oct-4-ene-1,8-diyl)(E)-dicarbamate (k) (0.135 g, 18% yield) as a black solid. ¹H NMR (499 MHz, Chloroform-d) δ 5.17 (dd, J=5.5, 2.6 Hz, 2H), 4.77 (s, 4H), 3.20-3.08 (m, 4H), 3.08-2.95 (m, 4H), 2.03 (td, J=9.3, 4.7 Hz, 2H), 1.66-1.56 (m, 4H), 1.43 (s, 36H), 1.39-1.32 (m, 4H); MS: mass calculated for C₃₂H₆₀N₄O₈: 628.44; found: positive: 629.9 (M+H)⁺, 652.0 (M+Na)⁺, 667.7 (M+K)⁺.

Optionally, gel permeation chromatography on LH20 GPC resin could be used for purification of the product k. The metathesis reaction stops progressing at ˜10-20% completion, with significant starting material di-tert-butyl (3-vinylpentane-1,5-diyl)dicarbamate in the reaction mixture. Optionally, the yield could be improved by isolating unreacted alkene after chromatography and treatment with charcoal and reusing it to make more of the product di-tert-butyl (3,6-bis(2-((tert-butoxycarbonyl)amino)ethyl)oct-4-ene-1,8-diyl)(E)-dicarbamate (k) in repeat metathesis reactions.

### G. Step 8. Synthesis of (E)-3,6-bis(2-ammonioethyl)oct-4-ene-1,8-diaminium) tetra-(2,2,2-trifluoroacetate) (1)

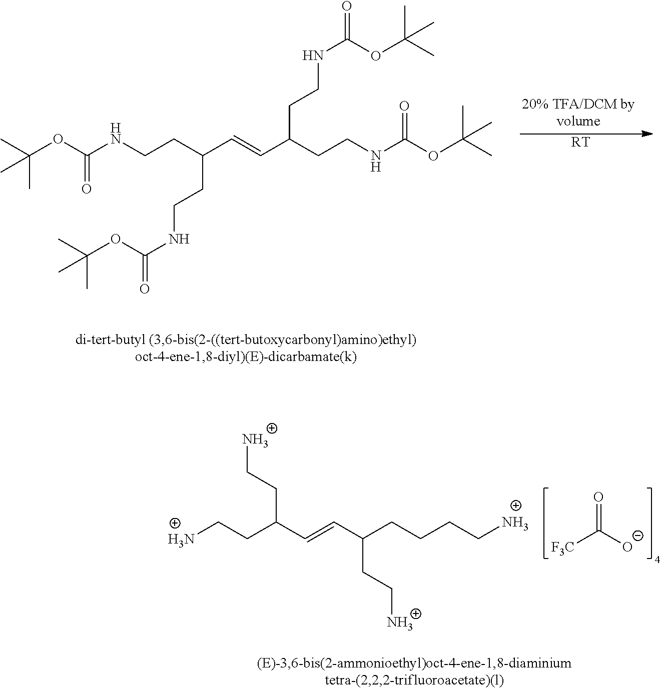

To a 0° C. solution of di-tert-butyl (3,6-bis(2-((tert-butoxycarbonyl)amino)ethyl)oct-4-ene-1,8-diyl)(E)-dicarbamate (k) (0.036 g, 0.057 mmol) dissolved in 1.7 ml of anhydrous dichloromethane was added trifluoroacetic acid (88 μl, 1.15 mmol) and the reaction was slowly warmed to room temperature over 16 hours under an inert atmosphere. The reaction was concentrated in vacuo and the resulting light brown solid dissolved in water. The water solution was filtered through a micro-syringe filter and the filtrate concentrated in vacuo to provide (E)-3,6-bis(2-ammonioethyl)oct-4-ene-1,8-diaminium) tetra-(2,2,2-trifluoroacetate) (1) (0.038 g, 97% yield) as an off-white solid. ¹H NMR (499 MHz, Deuterium Oxide) δ 5.44 (dd, J=5.6, 2.7 Hz, 2H), 3.12-2.92 (m, 8H), 2.37-2.22 (m, 2H), 1.94-1.80 (m, 4H), 1.79-1.67 (m, 4H).

### H. Step 9. Synthesis of Alkene Cryptate (m) (CCAOM4)

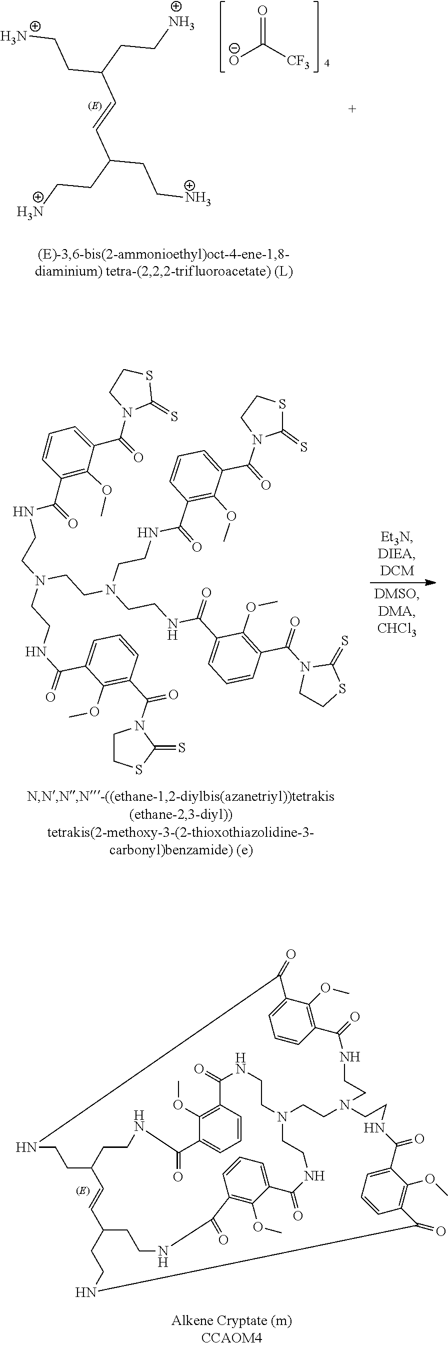

A solution of (E)-3,6-bis(2-ammonioethyl)oct-4-ene-1,8-diaminium) tetra-(2,2,2-trifluoroacetate) (1) (0.034 g, 0.050 mmol) dissolved in 1 ml of degassed dimethyl sulfoxide, 2 ml of degassed dimethylacetamide, and 22 ml of degassed chloroform was added 120 μl of degassed triethylamine, and 50 μl of diisopropylethylamine. Optionally and preferably, isopropanol is used in place of DMSO to dissolve the (E)-3,6-bis(2-ammonioethyl)oct-4-ene-1,8-diaminium) tetra-(2,2,2-trifluoroacetate). After sonication to form the free base ((E)-3,6-bis(2-ammonioethyl)oct-4-ene-1,8-diamine)), the solution was added simultaneously with a solution of N,N′,N″,N′″-((ethane-1,2-diylbis(azanetriyl))tetrakis(ethane-2,1-diyl))tetrakis(2-methoxy-3-(2-thioxothiazolidine-3-carbonyl)benzamide) (e) (0.067 g, 0.050 mmol) dissolved in 25 ml of degassed chloroform to a reaction vessel containing 98 μl of degassed triethylamine in 175 ml of degassed dichloromethane over a period of 7 days via two syringe pumps. On the 3^{rd} Day, 1.4 ml of degassed triethylamine was added to the reaction mixture. Once the additions were complete, the reaction was stirred for 16 hours at room temperature, and at reflux for 40 hours. Once cooled, the reaction was concentrated in vacuo and directly purified via flash chromatography (reverse phase C18, 15.5 g, 5-95% acetonitrile/25 mM ammonium acetate in water) to provide Alkene Cryptate (m, CCAOM4) (0.017 g, 31% yield) as a white solid. Due to the coexistence of several possible conformer/isomers characterization by ¹H-NMR proved challenging. The combination of HPLC and mass spectrometry was used instead. MS: mass calculated for C₅₈H₇₂N₁₀O₁₂: 1101.27; found: positive: 1102.00 (M+H)⁺, 1124.00 (M+Na)⁺. HPLC retention time=5.58 minutes (single peak).

HPLC Conditions: column: Agilent SB-C18 Poroshell 120, 4.6×150 mm, 2.7 μm, solvent A: 25 mM ammonium acetate in water, solvent B: acetonitrile, flow rate: 9 ml/min, gradient: 5% solvent B for 0.5 minutes, to 95% solvent B over 6 minutes, 95% solvent B for 2.5 minutes, to 5% solvent B over 1 minute.

Example II illustrates the synthesis of a phenolic alkene cryptate diacetic acid (o).

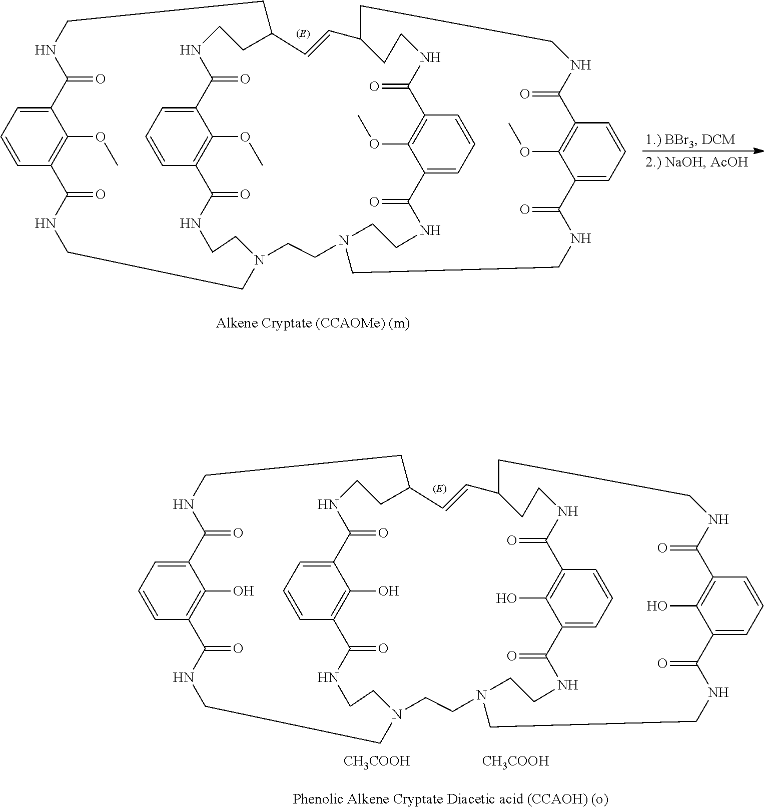

To a −10° C. solution of alkene cryptate (CCAOMe) (m) (0.012 g, 0.011 mmol) dissolved in 0.6 ml of anhydrous dichloromethane was added boron tribromide (0.45 ml, 1.0 M) in dichloromethane and the reaction slurry was stirred for 8 days at room temperature under an inert atmosphere. Due to evaporation, anhydrous dichloromethane was added each day to compensate for the loss of solvent to maintain the reaction concentration. The reaction was concentrated in vacuo, and co-evaporated three times with methanol to remove any remaining volatiles. The resulting residue was dissolved in 2 ml of 0.5 M sodium hydroxide (aq.) and filtered to remove solids. The pH of the combined filtrates was adjusted to ˜5 with concentrated acetic acid and the resulting white precipitate collected by centrifugation, and dried under vacuum to provide phenolic alkene cryptate diacetic acid (CCAOH4) (o) (0.012 g, 99% yield) as an off-white solid. The ¹H NMR in CD₃OD had too many overlapping signals to interpret, however this was simplified by using D₂O/NaOD. ¹H NMR (499 MHz, Deuterium Oxide) δ 7.85-7.69 (m, 5H), 7.68-7.55 (m, 3H), 6.48-6.35 (m, 4H), 5.79-5.67 (m, 1H), 5.34 (dd, J=15.3, 9.0 Hz, 1H), 3.72-3.37 (m, 8H), 3.37-3.01 (m, 8H), 2.97-2.72 (m, 12H), 1.97-1.78 (m, 2H), 1.78-1.57 (m, 4H), 1.36-1.19 (m, 4H). MS: mass calculated for C₅₄H₆₄N₁₀O₁₂: 1044.47 (free base); found: positive: 1045.80 (M+H)⁺, negative: 1043.80 (M−H)⁻. HPLC retention time=5.62-5.75 minutes (uneven doublet). TLC (10% methanol/dichloromethane): 2 spots R_{f}=0.05 & 0.10. Optionally, the CCAOH4 can be purified by adding TbCl₃ to convert to the terbium complex and purification using reverse phase HPLC or medium pressure reverse phase chromatography with 0.1% TFA in water and acetonitrile. Terbium complexes are more readily purified by reverse phase chromatography than uncomplexed ligands for CCAOH4 and derivatives.

HPLC Conditions: column: Agilent SB-C18 Poroshell 120, 4.6×150 mm, 2.7 μm, solvent A: 25 mM ammonium acetate in water, solvent B: acetonitrile, flow rate: 0.9 ml/min, gradient: 5% solvent B for 0.5 minutes, to 95% solvent B over 6 minutes, 95% solvent B for 2.5 minutes, to 5% solvent B over 1 minute.

Example III illustrates synthesis of a cryptate sulfonyl chloride: CCAOH—SO₂—Cl (p).

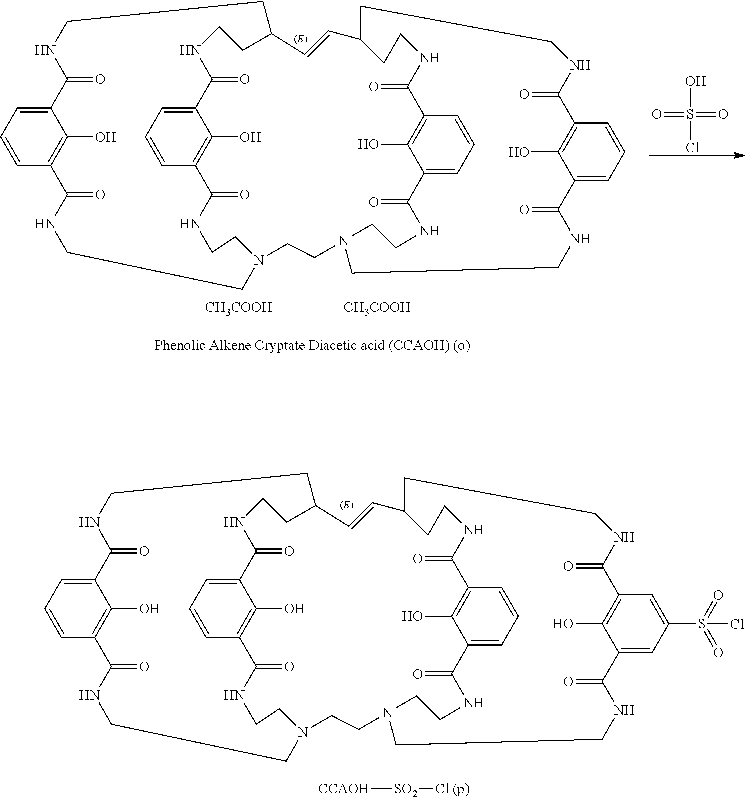

Chlorosulfonic acid (0.5 ml) was added to CCAOH (o) (5.0 mg, 4.78 μmol) at −10° C. and the reaction was stirred for 16 hours slowly warming to room temperature under an inert atmosphere. A portion of the reaction mixture was quenched by adding carefully to a solution of ice water to provide off-white precipitates (CCAOH—SO₂—Cl) (p) (˜1.0 mg, wet at pH=1, presumably containing HCl and H₂SO₄) which were collected by centrifugation and the water decanted. HPLC retention time=4.85-4.91 (doublet) and 5.16-5.21 (doublet) minutes. Optionally, the CCAOH terbium complex can be sulfonated by using a longer reaction time. The terbium is lost during this process.

Optionally and preferably, the sulfonyl chloride CCAOH₄—SO₂Cl is converted to the sulfonic acid CCAOH₄—SO₂OH through dissolution in aqueous NaOH (>1M) and incubating overnight at ambient temperature. After acidification to pH 1, TbCl₃ is added to form the CCAOH—SO₃H terbium complex and the CCAOH—SO₃H terbium complex is purified using reverse phase HPLC or medium pressure with 0.1% TFA in water and acetonitrile. Terbium complexes are more readily purified by reverse phase chromatography than uncomplexed ligands for CCAOH4 and derivatives. The material is treated with excess oxalyl chloride in DMF to form the sulfonyl chloride CCAOH₄—SO₂Cl. This material can then be treated with an excess of amine (for example, t-butyl glycinate) to form a cryptate sulfonamide CCAOH₄—SO₂NHR and an oxalylamide derivative as a side product. The sulfonamide can be treated with TbCl₃ to reintroduce terbium and purified using gradient reverse phase HPLC or medium pressure with 0.1% TFA in water and acetonitrile as the 2 eluants (for example, making CCAOH₄—SO₂NHCH₂COO-t-Bu terbium complex. CCAOH₄—CCAOH₄—SO₂NHCH₂COO-t-Bu terbium complex is hydrolyzed to CCAOH₄—SO₂NHCH₂COOH terbium complex. CCAOH₄—SO₂NHCH₂COOH terbium complex made in this way showed a fluorescence spectrum close to CCAOH₄ Terbium complex. It apparently lost Tb during MALDI mass spectroscopy, exhibiting peaks for the terbium-free CCAOH₄—SO₂NHCH₂COOH as the M+H, M+Na, and M+K peaks in MALDI mass spectroscopy.

Optionally the sulfonic acid CCAOH₄—SO₃H for the method outlined above is made by treating CCAOH with fuming sulfuric acid.

Optionally, in the method outlined above, the oxalyl chloride/DMF is replaced with trichloro-triazene, POCl₃, or another sulfonic acid activating agent to convert CCAOH4SO₃H to CCAOH4SO₂Cl or another activated sulfonamide.

Optionally, in the method outlined above, if the sulfonamide contains an unactivated ester, the crude unactivated ester can be hydrolyzed to carboxylic acid, treated with TbCl₃, and purified using gradient reverse phase HPLC or medium pressure reverse phase chromatography with 0.1% TFA in water and acetonitrile as the 2 eluants. For example, the t-butyl-glycine sulfonamide CCAOH₄—SO₂NHCH₂COO-t-Bu can be converted to CCAOH₄—SO₂NHCH₂COOH Tb complex and purified in this way.

The following structures are shown below:

| Structure | Name |
|---|---|
| 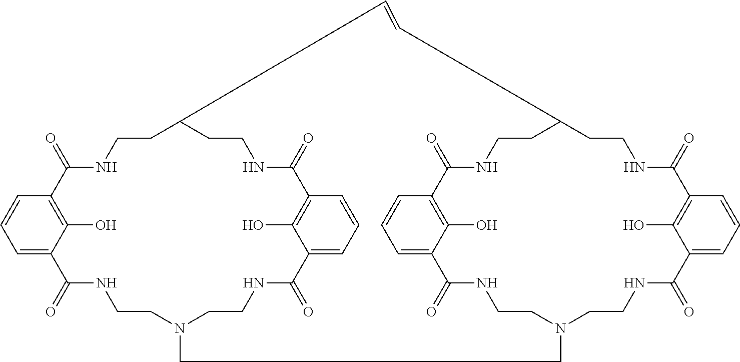 [SMILES: O=C1NCCC2/C=C/C3CCNC(=O)c4cccc(c4O)C(=O)NCCN(CCNC(=O)c4cccc(c4O)C(=O)NCC3)CCN(CCNC(=O)c3cccc1c3O)CCNC(=O)c1cccc(c1O)C(=O)NCC2] | CCAOH₄ |
| 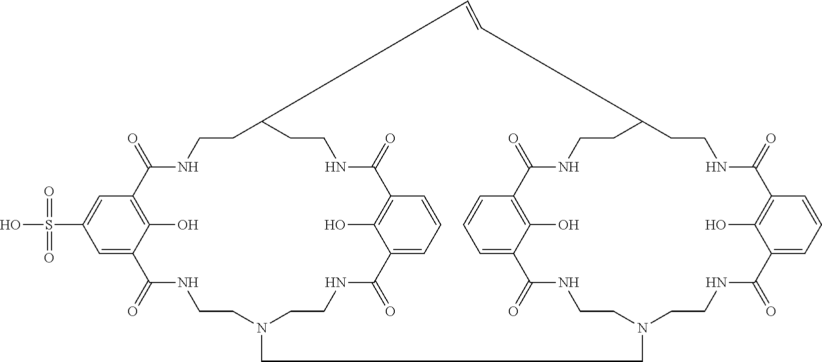 [SMILES: O=C1NCCC2/C=C/C3CCNC(=O)c4cccc(c4O)C(=O)NCCN(CCNC(=O)c4cc(S(=O)(=O)O)cc(c4O)C(=O)NCC3)CCN(CCNC(=O)c3cccc1c3O)CCNC(=O)c1cccc(c1O)C(=O)NCC2] | CCAOH₄—SO₃H |
| 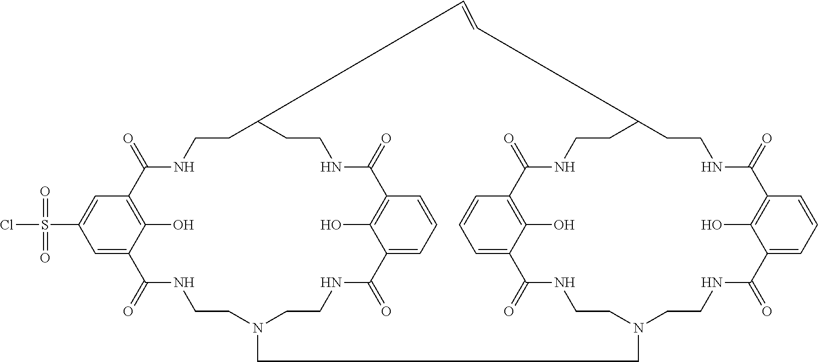 [SMILES: O=C1NCCC2/C=C/C3CCNC(=O)c4cccc(c4O)C(=O)NCCN(CCNC(=O)c4cc(S(=O)(=O)Cl)cc(c4O)C(=O)NCC3)CCN(CCNC(=O)c3cccc1c3O)CCNC(=O)c1cccc(c1O)C(=O)NCC2] | CCAOH₄—SO₂Cl |
| 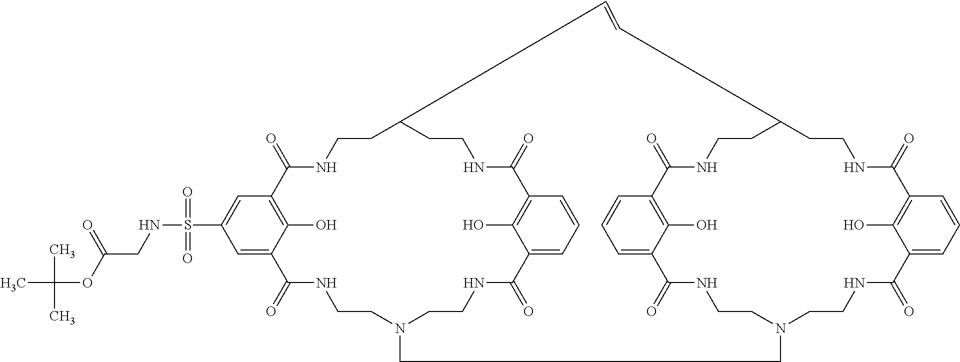 [SMILES: CC(C)(C)OC(=O)CNS(=O)(=O)c1cc2c(O)c(c1)C(=O)NCCN1CCNC(=O)c3cccc(c3O)C(=O)NCCC(/C=C/C3CCNC(=O)c4cccc(c4O)C(=O)NCCN(CCNC(=O)c4cccc(c4O)C(=O)NCC3)CC1)CCNC2=O] | CCAOH₄—O₂NHCH₂COO—t-Bu |
| 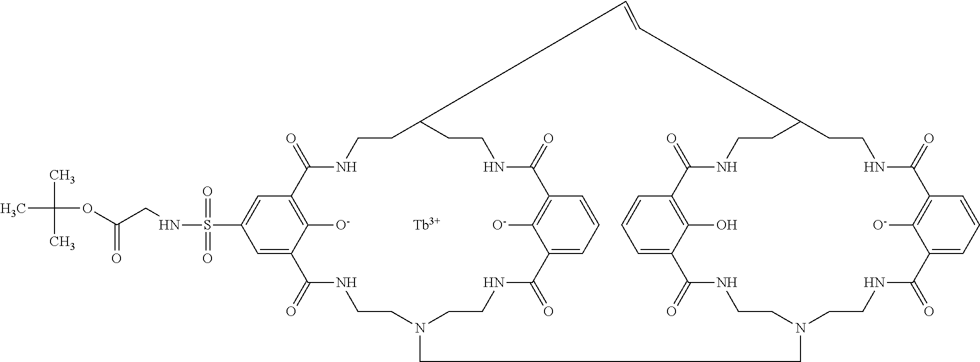 | CCAOH₄—SO₂NHCH₂COOBu^{t} Tb complex |
| 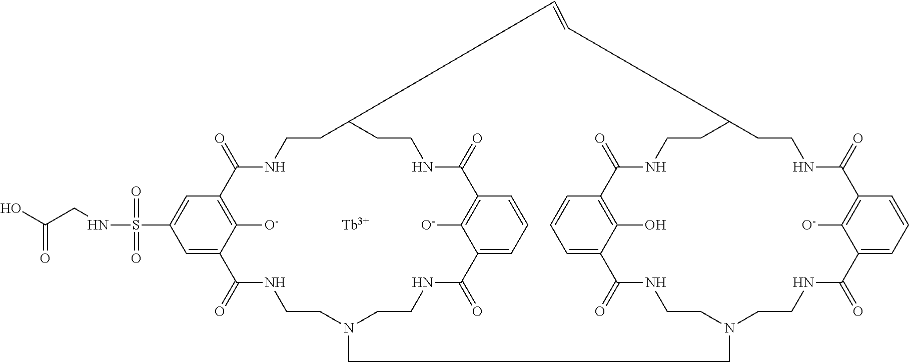 | CCAOH₄—SO₂NHCH₂COOH Tb complex |
| 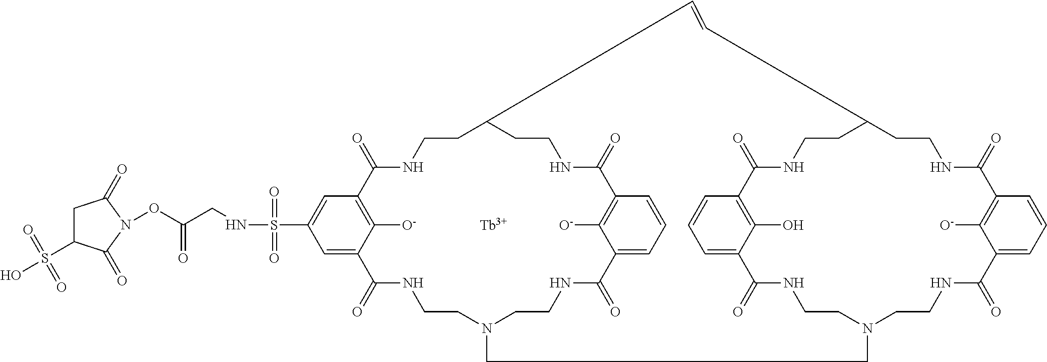 | CCAOH₄—SO₂NHCH₂COsNHS Tb complex |

LCMS analysis: mass calculated for C₅₄H₆₃ClN₁₀O₁₄S: 1142.39 (free base); found: positive: 1125.73 (M−Cl+HOH)⁺, observed is the sulfonic acid caused by hydrolysis on the column. This is further supported by the experiment below.

HPLC Conditions: column: Agilent SB-C18 Poroshell 120, 4.6×150 mm, 2.7 μm, solvent A: 25 mM ammonium acetate in water, solvent B: acetonitrile, flow rate: 0.9 ml/min, gradient: 5% solvent B for 0.5 minutes, to 95% solvent B over 6 minutes, 95% solvent B for 2.5 minutes, to 5% solvent B over 1 minute. Optionally, better performance on analytical HPLC is obtained by using gradient reverse phase HPLC or medium pressure with 0.1% TFA in water and acetonitrile as the 2 eluants.

The formation of the sulfonyl chloride was verified using the following reaction (Synthesis of CCAOH—SO₂—NH-Cy (q)):

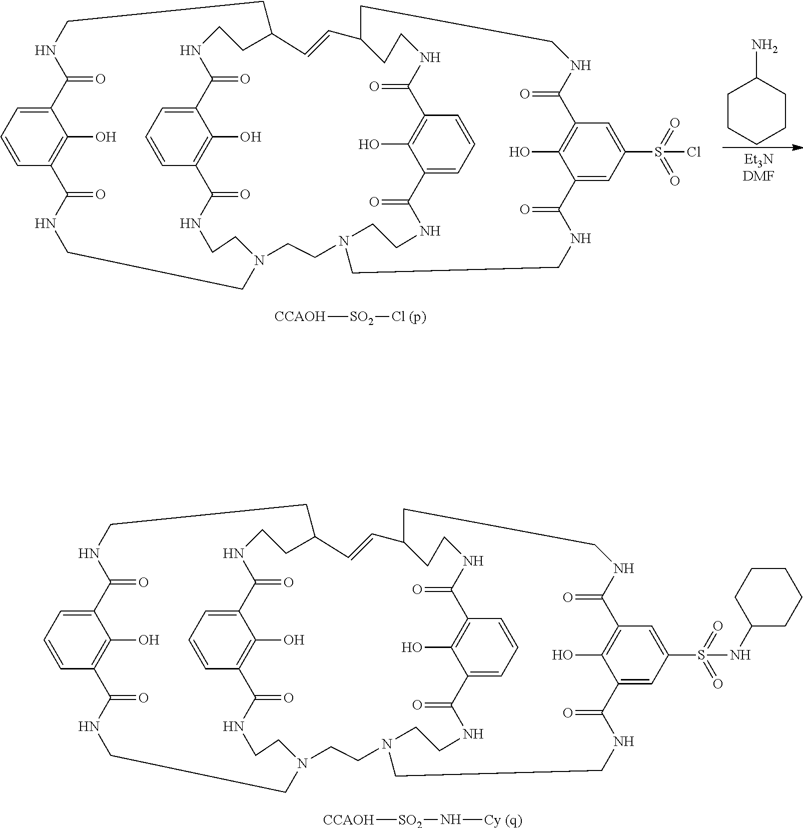

To a 0° C. solution of cyclohexylamine (50 μl) was added freshly prepared CCAOH—SO₂—Cl (q) (1.0 mg, 0.874 μmol) to form a white solid. To the solids was added 0.3 ml of anhydrous dimethylformamide and the suspension was stirred at room temperature in a sealed vial under an inert atmosphere for 16 hours. A small aliquot was removed, dissolved in DMSO, and used for analysis. HPLC retention time of a new peak=5.87-5.92 (doublet) minutes.

LCMS analysis: mass calculated for C₆₀H₇₅N₁₁O₁₄S: 1205.52 (free base); found: positive: 1206.87 (M+H)⁺.

HPLC Conditions: column: Agilent SB-C18 Poroshell 120, 4.6×150 mm, 2.7 μm, solvent A: 25 mM ammonium acetate in water, solvent B: acetonitrile, flow rate: 0.9 ml/min, gradient: 5% solvent B for 0.5 minutes, to 95% solvent B over 6 minutes, 95% solvent B for 2.5 minutes, to 5% solvent B over 1 minute.

Example IV illustrates the synthesis of alternative route to a sulfonyl chloride.

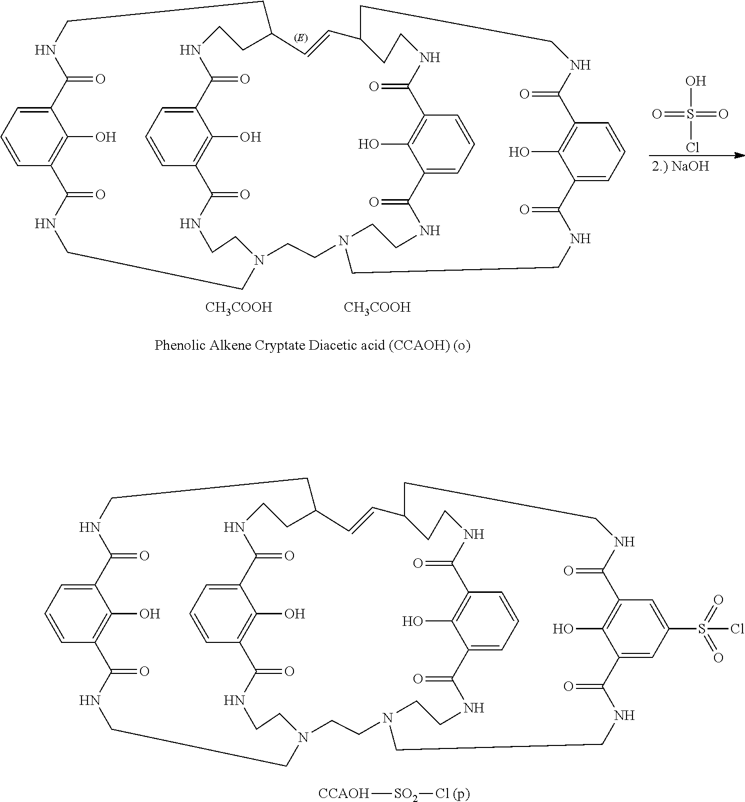

Chlorosulfonic acid (0.8 ml) was added to CCAOH (o) (8.0 mg, 7.65 μmol) at −10° C. and the reaction was stirred for 40 hours slowly warming to room temperature under an inert atmosphere. The reaction mixture was quenched by adding carefully to a solution of ice water to provide off-white precipitates. The pH of the solution was adjusted to 10 with 6.0 M sodium hydroxide (aq.) and stirred for 30 minutes. The pH was then, adjusted to 5 with 1.0 M hydrochloric acid (aq.), and the entire solution loaded onto a reversed-phase C18 column and eluted (0-95% methanol/water (containing 0.1% trifluoroacetic acid)) to provide CCAOH—SO₂—OH (˜8.0 mg) as a white solid. LCMS analysis: mass calculated for C₅₄H₆₄N₁₀O₁₅S: 1124.43 (free acid); found: positive: 1125.73 (M+H)⁺.

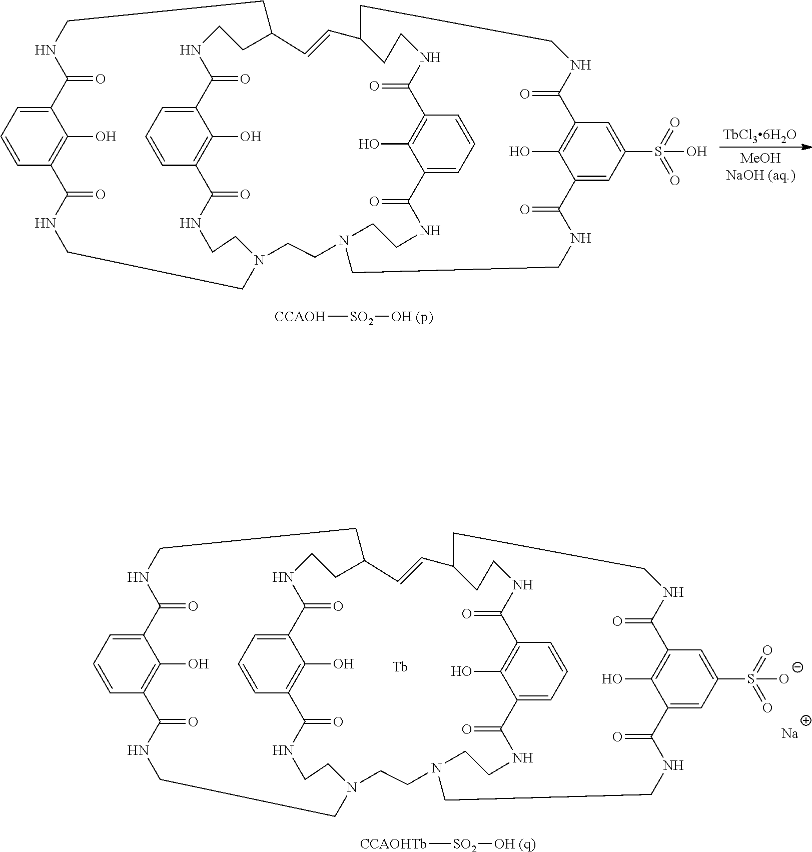

To a solution of sodium CCAOH—SO₂—OH (DJS-14-198) (p) (8.0 mg, 7.11 μmol) dissolved in 4.0 ml of methanol was added terbium(III)chloride hexahydrate (1.40 ml, 0.005 M) dissolved in methanol, 30 μl of 1 M sodium hydroxide (aq.), and the reaction was heated at 60° C. for 30 minutes under an inert atmosphere. The reaction was concentrated in vacuo and used in the subsequent step without further purification.

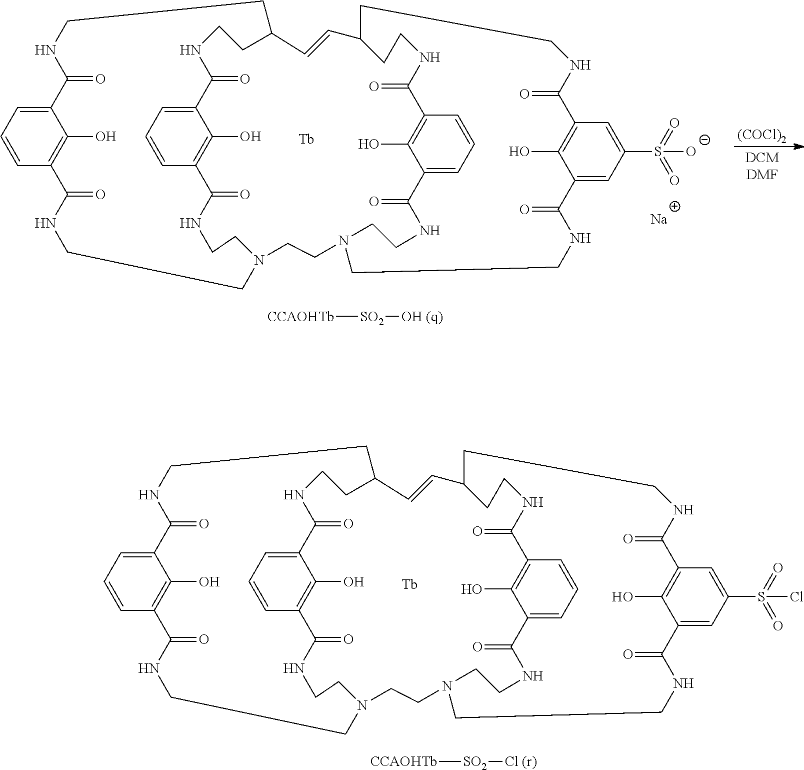

To a solution of sodium CCAOHTb-SO₂—OH (DJS-14-201) (q) (8.0 mg, 6.23 μmol) dissolved in 2.0 ml of dichloromethane and 5.0 μl of anhydrous dimethylformamide was added oxalyl chloride (8.0 μl, 93 μmol), and the reaction was stirred for 2 hours at room temperature under an inert atmosphere. The reaction was concentrated in vacuo and used in the subsequent step without further purification.

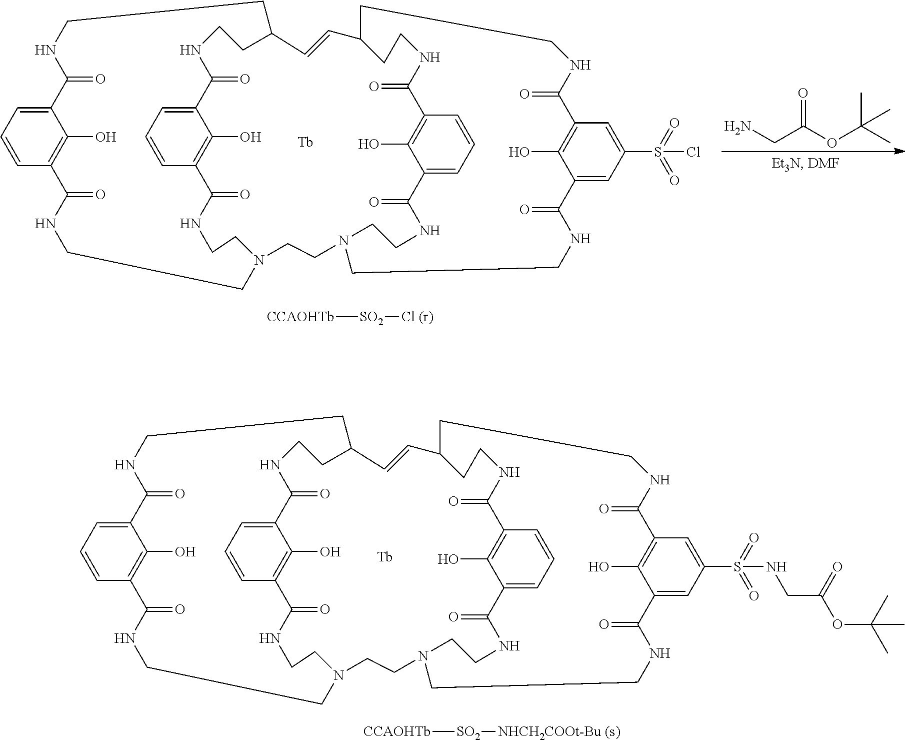

A solution of triethylamine (15 μl, 0.104 mmol) and tert-butyl glycinate (17 μl, 0.123 mmol) dissolved in 2.0 ml of anhydrous dimethylformamide was added to CCAOHTb-SO₂—Cl (DJS-14-202) (r) (8.0 mg, 6.14 μmol), and the reaction was stirred for 4 hours at room temperature under an inert atmosphere. The reaction was concentrated in vacuo and used in the subsequent step without further purification.

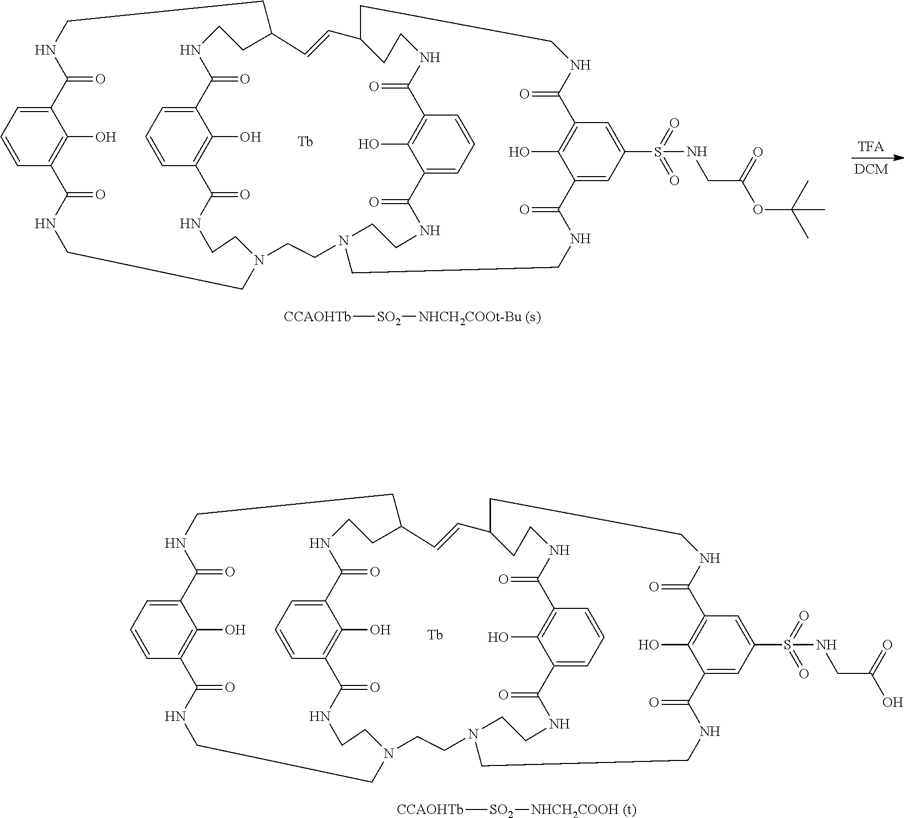

To a solution of CCAOHTb-SO₂—NHCH₂COOH (DJS-14-203) (8.0 mg, 5.73 μmol) dissolved in 2.0 ml of anhydrous dichloromethane was added trifluoroacetic acid (0.40 ml, 5.27 mmol) and the reaction was stirred for 16 hours at room temperature under an inert atmosphere. The reaction was concentrated in vacuo, dissolved in DMSO/water, and directly purified via flash chromatography (reverse-phase C18, 10-80% methanol/water containing 0.1% trifluoroacetic acid) to provide CCAOHTb-SO₂—NHCH₂COOH (t) (14-204p2, 1.5 mg) and a second sample (14-204p3, 3.4 mg) which was further treated with TFA/DCM to give CCAOHTb-SO₂—NHCH₂COOH (t) (4.9 mg (as two samples), 55% yield over 5 steps) as a light yellow oil which retains its' green fluorescence under UV. MALDI: mass calculated for C₅₆H₆₇N₁₁O₁₆S: 1181.45 (metal free complex); found: positive: 1182 (M+H)⁺, 1204 (M+Na)⁺, 1220 (M+K)⁺. Metal complexes did not ionize in MALDI/TOF, and were therefore not observed.

HPLC Conditions: column: Agilent SB-C18 Poroshell 120, 4.6×150 mm, 2.7 μm, solvent A: 25 mM ammonium acetate in water, solvent B: acetonitrile, flow rate: 0.9 ml/min, gradient: 5% solvent B for 0.5 minutes, to 95% solvent B over 6 minutes, 95% solvent B for 2.5 minutes, to 5% solvent B over 1 minute.

Example V illustrates the fluorescent properties of an alkene cryptate (m) of the present invention.

The fluorescent properties of alkene cryptate (m) prepared in accordance to Example I were analyzed. Lumi4™ cryptate commercially available from Cisbio was used as a comparative cryptate in this example. In each instance, 3.8 mg of the cryptate was dissolved in 3.8 mL methanol to a concentration of 1 mg/mL (0.83 mM). Separately, a 2 mg/mL (5.35 mM) solution of TbCl₃ in 5 mM citrate (pH 5.13) was prepared. Terbium-cryptate complexes were obtained by mixing the 2 mg/mL (5.35 mM) solution of TbCl₃ to a 1.5-fold molar excess in 1 mL of cryptate solution.

Qualitative fluorescence was analyzed in a dark room using ultraviolet (UV) light filtered at 365 nm for excitation. TbCl₃ was not visibly fluorescent under these conditions. While alkene cryptate (m) alone emitted visibly blue background fluorescence, Tb-ion complexed alkene cryptate (m) produced visibly green fluorescence. Similarly, Tb-ion complexed Lumi4™ produced visibly green fluorescence, although the color in this sample was a deeper green.

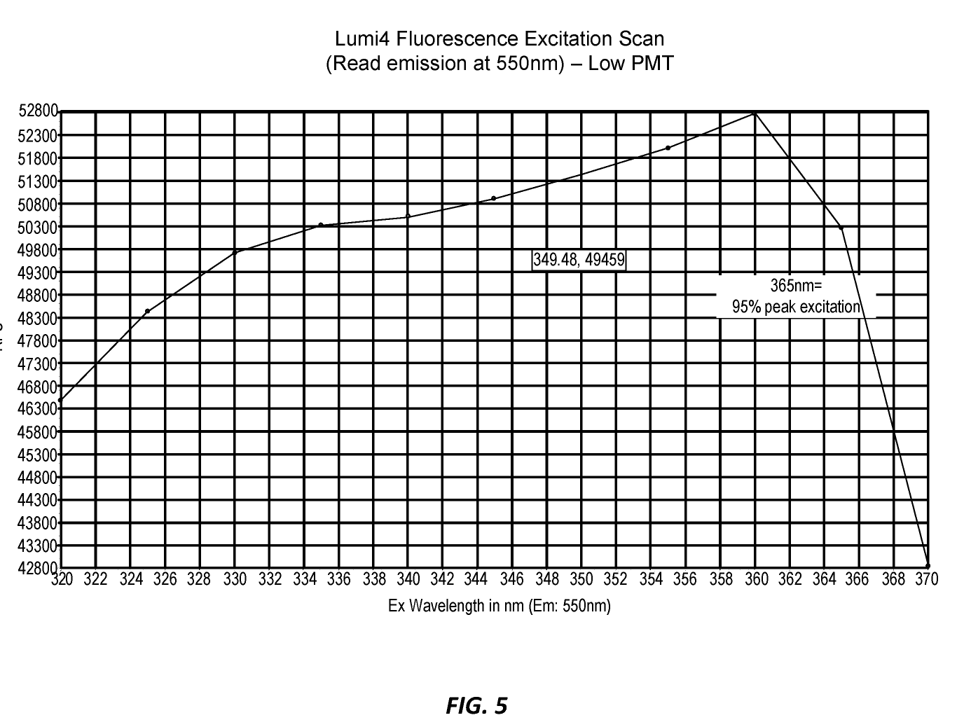

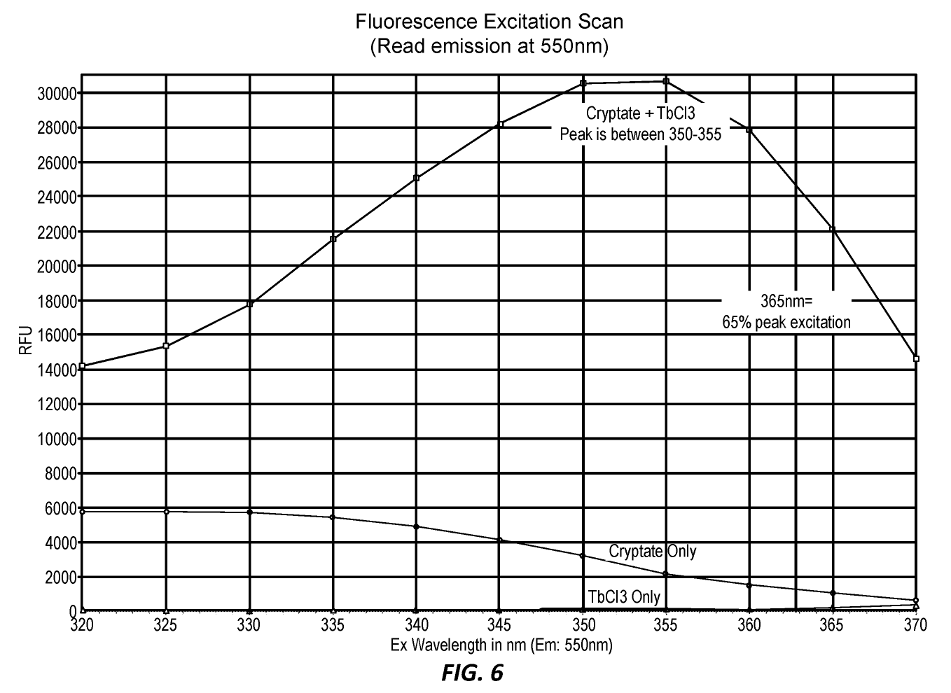

Terbium complexes typically produce characteristic fluorescence emission peaks of about 490, 550, 580, and 620 nm upon excitation at 365 nm, with the 550 nm peak being the prominent peak. The emission spectra of the comparative example Lumi4™ complexed with TbCl₃ (“Lumi4™-Tb”) was analyzed for optimal excitation wavelengths for maximum emission at 550 nm. Results showed excitation at 365 nm produced maximum fluorescence emission at 550 nm in Lumi4™-Tb (FIG. 5). When alkene cryptate (m) complexed with TbCl₃ (“alkene cryptate (m)-Tb”) was analyzed for optimal excitation wavelengths, maximum emission at 550 nm was observed at excitation wavelengths between 350-355 nm (FIG. 6). Fluorescence of complexed alkene cryptate (m)-Tb upon excitation at 350-355 nm was extensively greater than for TbCl₃ alone, although uncomplexed alkene cryptate (m) produced some background fluorescence.

These results show that terbium-ion alkene cryptate (m) has significant fluorescence at 550 nm with an excitation wavelength of 365 nm. This shows that emission at 550 nm is preferably achieved at excitation wavelengths of 350-355 and 365 nm for alkene cryptate (m) and Lumi4™ complexed cryptates, respectively.

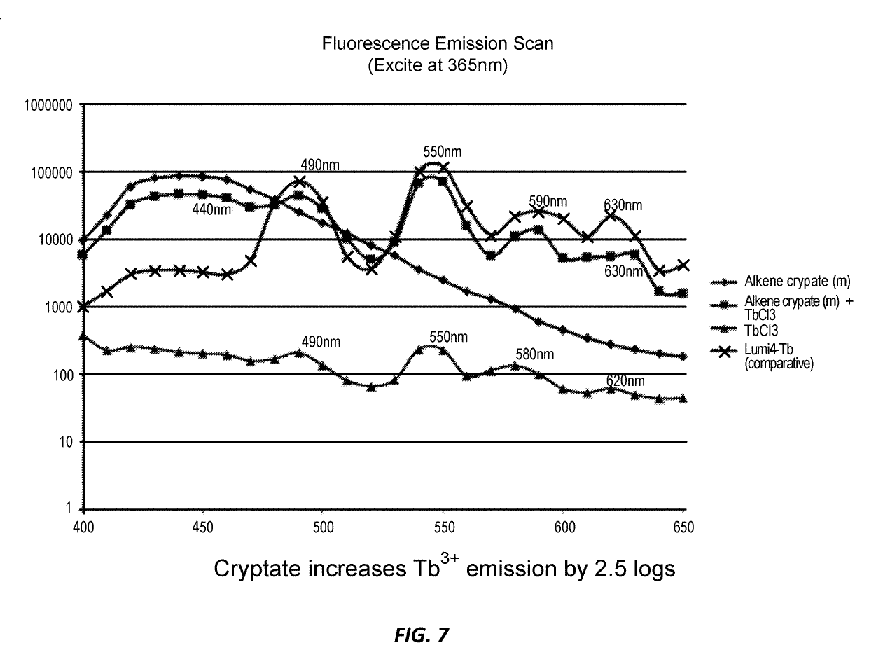

Fluorescence emission spectra of the inventive and comparative cryptates were directly compared at an excitation wavelength of 365 nm (FIG. 7). Data were recorded over the broad emission bandpass of 400-650 nm. Terbium alone, provided in the form of TbCl₃, expectedly produced characteristic emission peaks at about 490, 550, 580, and 620 nm. Significantly, terbium complexed with the inventive cryptate increased terbium fluorescence emission by about 2.5 logs.

Both cryptate complexes produced emission spectra having characteristic terbium emission peaks at about 490, 550, 580, and 620 nm, although the fourth peak was slightly shifted to 630 nm in the alkene cryptate (m)-Tb sample. An additional shoulder peak was observed at about 440 nm in the alkene cryptate (m)-Tb sample, which may have been due to background fluorescence of the cryptate itself. Slightly lower emission of the inventive sample at 550 nm was likely due to excitation at 365 nm, which is optimal for emission of Lumi4™-Tb (FIG. 5), but suboptimal for cryptate-Tb (FIG. 6). Collectively, these results show that alkene cryptate (m) significantly increases terbium fluorescence to levels comparative to Lumi4™ while maintaining characteristic terbium spectral patterns.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A compound of Formula I: 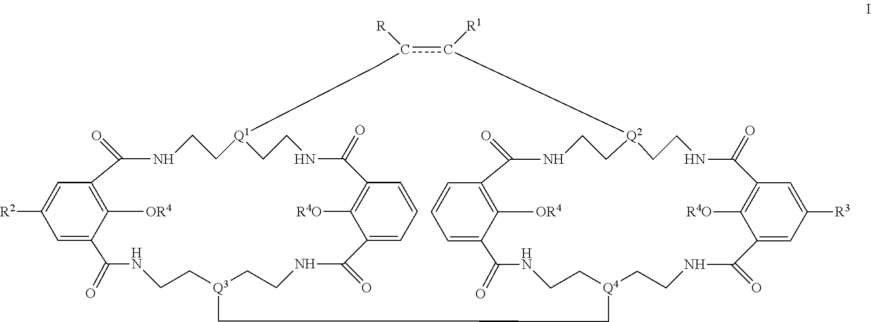
wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and R¹ join to form an optionally substituted cyclopropyl group wherein the dotted bond is absent;
R² and R³ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, or an activated group that can be linked to a biomolecule, wherein the activated group is a member selected from the group consisting of a halogen, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group and L;
R⁴ are each independently a hydrogen, C₁-C₆ alkyl, or alternatively, 3 of the R⁴ groups are absent and the resulting oxides are chelated to a lanthanide cation; and
Q¹-Q⁴ are each independently a member selected from the group consisting of carbon and nitrogen.

2. The compound of claim 1, wherein R and R¹ are each hydrogen.

3. The compound of claim 1, wherein at least one of R² and R³ is —SO₂Cl.

4. The compound of claim 1, wherein at least one of R² and R³ is a member selected from the group consisting of hydrogen, activated acyl, optionally substituted alkylsulfonate ester, optionally substituted arylsulfonate ester, optionally substituted amino, formyl, glycidyl, halo, haloacetamidyl, haloalkyl, hydrazinyl, imido ester, isocyanato, isothiocyanato, maleimidyl, mercapto, carboxyl, alkoxycarbonyl, amido, sulfonato, alkoxycarbonylalkyl, and alkoxyalkyl.

5. The compound of claim 1, wherein at least one of R² and R³ is a member selected from the group consisting of activated acyl, optionally substituted alkylsulfonate ester, optionally substituted arylsulfonate ester, formyl, glycidyl, halo, haloacetamidyl, haloalkyl, imido ester, isocyanato, isothiocyanato, and maleimidyl.

6. The compound of claim 1, wherein at least one of R² and R³ a member selected from the following group: 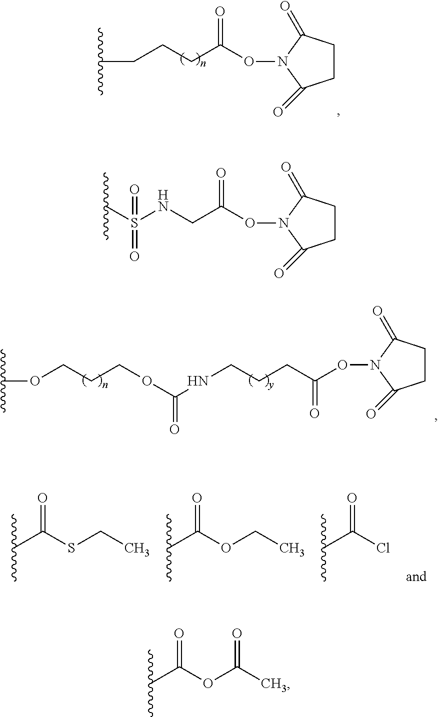
wherein n and y are each independently selected from 0 to 5.

7. The compound of claim 1, wherein the compound of Formula I has a chelated lanthanide cation.

8. The compound of claim 1, wherein the compound of Formula I has the structure:

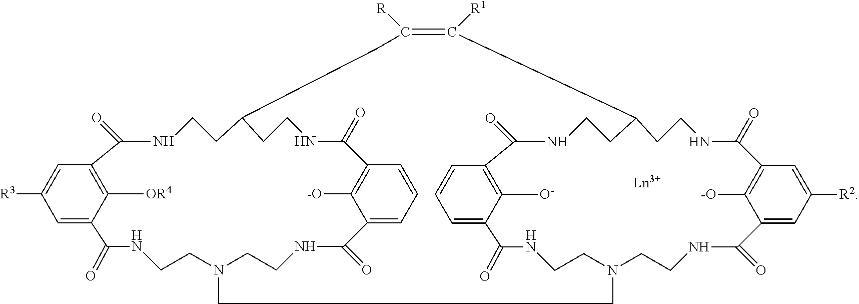

9. The compound of claim 1, wherein the lanthanide ion is a member selected from the group consisting of neodymium (Nd), samarium (Sm), europium (Eu), terbium (Tb), dysprosium (Dy) and ytterbium (Yb).

10. The compound of claim 1, wherein the lanthanide ion Ln³⁺ is selected from the group consisting of europium and terbium.

11. The compound of claim 1, wherein the lanthanide ion is terbium.

12. A bioconjugate compound of Formula II: 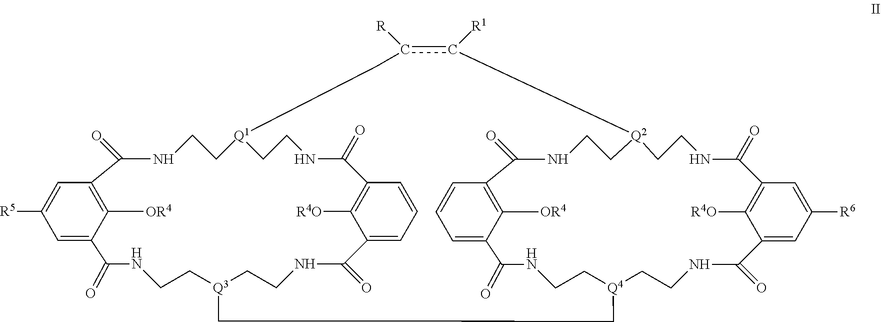
wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and R¹ join to form an optionally substituted cyclopropyl group wherein the dotted bond is absent;
R⁵ and R⁶ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group and -L^{q}-B, wherein -L^{q}-B comprises a linking group and a biomolecule, wherein the compound comprises at least one -L^{q}-B;
R⁴ are each independently a hydrogen, C₁-C₆ alkyl, or alternatively, 3 of the R⁴ groups are absent and the resulting oxides are chelated to a lanthanide cation; and
Q¹-Q⁴ are each independently a member selected from the group consisting of carbon and nitrogen.

13. The compound of claim 12, wherein B is an antibody, protein or peptide.

14. The compound of claim 12, wherein L^{q} comprises a member selected from the group consisting of amide, ether, thioether, ester, thioester, carbamate, urea, and thiourea.

15. The compound of claim 12, wherein the compound of Formula II has the following structure:

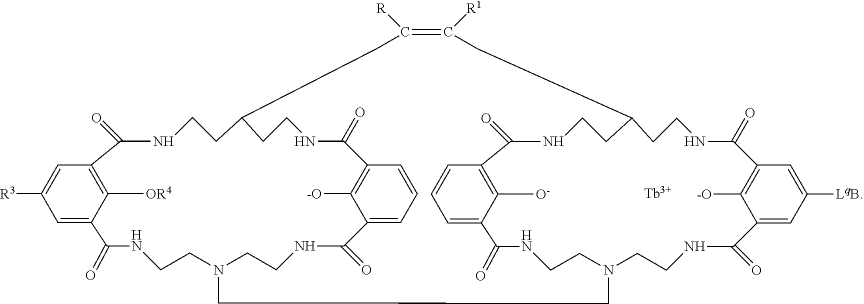

16. An assay method for detecting an analyte in solution, the method comprising:
contacting a sample with a compound of Formula II comprising a lanthanide ion, wherein the compound of Formula II binds with an analyte;
exciting the sample with light; and
detecting fluorescence emission to detect the analyte.

17. The assay method of claim 16, wherein the compound of Formula II has the following structure: 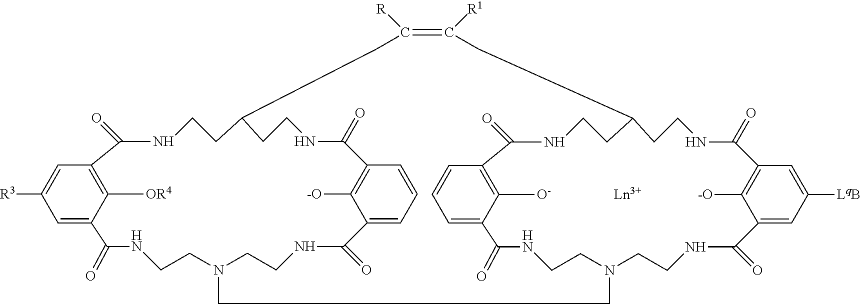
wherein B binds an analyte having an acceptor fluorophore; and
detecting fluorescence emission from the acceptor fluorophore.

18. The assay method of claim 16, wherein the lanthanide cation is a member selected from the group consisting of europium and terbium.

19. The assay method of claim 16, wherein the excitation is with UV light.

20. The assay method of claim 16, wherein the wavelength of excitation is between about 300 nm to about 400 nm.

21. The assay method of claim 16, wherein the acceptor fluorophore emits in the visible to near IR.

22. The assay method of claim 16, wherein the visible to near IR is between 500 and 800 nm.
